(19) United States
(12) Patent Application Publication
(10) Pub. No.: US 2011/0098548 A1
(43) Pub. Date: Apr. 28, 2011

(54) METHODS FOR MODELING INSULIN THERAPY REQUIREMENTS

(75) Inventors: Erwin S. Budiman, Fremont, CA (US); Nathan Crouther, San Francisco, CA (US); Tim Dunn, San Francisco, CA (US); Gary Hayter, Oakland, CA (US); Ramiro Palma, Austin, TX (US); Marc B. Taub, Mountain View, CA (US)

(73) Assignee: ABBOTT DIABETES CARE INC., Alameda, CA (US)

(21) Appl. No.: 12/910,620

(22) Filed: Oct. 22, 2010

Related U.S. Application Data
(60) Provisional application No. 61/254,156, filed on Oct. 22, 2009.

(51) Int. Cl. A61B 5/145

(52) U.S. Cl. 600/365

(57) ABSTRACT

Various methods for improving the use of model based prediction of future blood glucose control in a patient having diabetes are described. A system for processing diabetes related information, including glucose information, for accurately predicting future glucose levels as a function of glucose data, carbohydrate intake, insulin delivery history and exercise history and then providing recommendations related to the predicted future glucose levels, is also described.

### CROSS-REFERENCES TO RELATED APPLICATIONS

This application claims the benefit of U.S. Application No. 61/254,156, filed Oct. 22, 2009, which is incorporated herein by reference in its entirety.

### BACKGROUND

Diabetes is a metabolic disorder that afflicts tens of millions of people throughout the world. Diabetes results from the inability of the body to properly utilize and metabolize carbohydrates, particularly glucose. Normally, the finely tuned balance between glucose in the blood and glucose in bodily tissue cells is maintained by insulin, a hormone produced by the pancreas which controls, among other things, the transfer of glucose from blood into body tissue cells. Upsetting this balance causes many complications and pathologies including heart disease, coronary and peripheral artery sclerosis, peripheral neuropathies, retinal damage, cataracts, hypertension, coma, and death from hypoglycemic shock.

In patients with insulin-dependent diabetes, the symptoms of the disease can be controlled by administering additional insulin (or other agents that have similar effects) by injection or by external or implantable insulin pumps. The “correct” insulin dosage is a function of the level of glucose in the blood. Ideally, insulin administration should be continuously readjusted in response to changes in blood glucose level.

Patients typically monitor their blood glucose levels using finger-stick style glucose monitors. Systems are also available for monitoring blood glucose levels by implanting a glucose sensitive probe into the patient. Such probes measure various properties of blood or other tissues, including optical absorption, electrochemical potential and enzymatic products. The output of finger-stick glucose monitors or probe sensors can be communicated to a hand held device that is used to calculate an appropriate dosage of insulin to be delivered into the blood stream in view of several factors, such as a patient's present glucose level, insulin usage rate, carbohydrates consumed or to be consumed and exercise, among others. These calculations can then be used to determine the amount of insulin to be injected or they may be used to control a pump that delivers the insulin, either at a controlled “basal” rate, or as a “bolus.” When provided as an integrated system, the continuous glucose monitor, controller and pump work together to provide continuous glucose monitoring and insulin pump control.

As stated above, such systems at present require intervention by a patient to calculate and control the amount of insulin to be delivered. However, there may be periods when the patient is not able to adjust insulin delivery. For example, when the patient is sleeping, he or she cannot intervene in the delivery of insulin, yet control of a patient's glucose level is still necessary. A system capable of integrating and automating the functions of glucose monitoring and controlled insulin delivery would be useful in assisting patients in maintaining their glucose levels, especially during periods of the day when they are unable to intervene.

What has been needed, and heretofore unavailable, is a system that uses available glucose and meal, insulin injection and exercise event information and models a patient's present and future blood glucose levels from that information so as to allow the patient to control his or her blood glucose levels. Such a system would include various features to optimize the model to ensure a sufficient correspondence between model estimated values and actual glucose data to allow the model estimated values to be used to control the delivery of insulin to more effectively control a patient's blood glucose level. Moreover, such a system may include functions designed to assist in diagnosing a patient's disease state, and determining the emptying rate of the patient's gastric system, among other useful functions. The present invention satisfies these and other needs.

### SUMMARY OF THE INVENTION

Briefly, and in general terms, the invention is directed to new and improved systems and methods for management of blood glucose level management, including systems and methods for improving the usability and safety of systems including continuous glucose monitors and drug delivery pumps.

In one general aspect, the invention defines a specific type of model used for a bolus calculator and therapy calculator, and potential ways to fit the model with actual data. In an other aspect, a general model is constructed that incorporates assumptions to simplify the model to reduce the calculation burden on a processor that is programmed using suitable software commands to carry out the model process.

In yet another aspect, the various aspects of the invention may be incorporated into a computation device, which may be either static, such as a personal computer or server, or may be mobile, such as a specially designed hardware device, PDA, handheld device, cell phone and the like. In such an aspect, the models and processes of various aspects of the invention retrieve or access data pertinent to control of a patient's glucose level, such as meal data, insulin delivery/administration data, and a patient's past and present glucose values; this data is then analyzed to provide recommendations to the patient regarding timing and amount of insulin that will be needed to keep the patient's glucose level within a desired range. In still another aspect, the recommendations may be used to either prompt the patient to inject insulin, or program an insulin pump with the recommendations. In a further aspect, the recommendations may be communicated directly to the pump to program the pump to administer insulin in accordance with the recommendations.

In a still further aspect, the recommendations may include various parameters that relate to the administration of insulin, or alternatively, to other actions, such as a prompt to consume a mass of carbohydrates to prevent or counter the onset of hypoglycemia. In even further aspects, the recommendations may include recommendations to split a single large bolus into multiple boluses delivered over time. In some aspects, the time of the multiple boluses may be delayed a pre-determined period of time, or the patient may be prompted before the next bolus is given to measure his or her glucose level.

In still another aspect, the invention includes commanding the processor to update the model and identified parameters of the model at intervals as new glucose level data becomes available, and also provide updated recommendations to the patient based on the updates.

In still another aspect, the invention includes a method for predicting future blood glucose values from blood glucose data collected over time for a patient, comprising: measuring blood glucose data at selected times over a selected sampling period; analyzing the blood glucose data to determine selected patient specific parameters used to develop a model of the patient's blood glucose reaction to insulin therapy, carbohydrate intake and exercise; such that the blood glucose values as a function of time predicted by the model sufficiently predicts the measured blood glucose data to allow accurate estimation of the patient's future blood glucose values.

In yet another aspect, the step of analyzing is carried out by a processor under control of suitable software programming commands. In another aspect, the model used is an extended version of the Bergman Minimal Model. In still another aspect, the model is set up using a pseudo-steady state assumption to simply the calculation requirements of the model.

In a further aspect, the model includes determining insulin effectiveness as a function of insulin sensitivity and dosage size. In a still further aspect, the output of the model is transformed into physiologically meaningful parameters including insulin pharmacokinetics, insulin pharmacodynamics, residual beta cell function, liver function, gastric function, and counter-regulatory response to low blood and exercise-induced glucagon secretion.

In yet another aspect, the invention also includes determining the patient's disease state using the physiologically meaningful parameters. In another aspect, the invention includes providing data related to events such as carbohydrate intake, insulin dosage and duration and intensity of exercise; temporally weighting such data; and using the temporally weighted data to improve the fit of the model to the measured blood glucose data.

In still another aspect, the invention includes providing data related to events such as carbohydrate intake, insulin dosage and duration and intensity of exercise; temporally shifting such data; and using the temporally shifted data to improve the fit of the model to the measured blood glucose data.

In another aspect, the model is simplified using selected assumptions regarding selected data to reduce the time needed to determine the selected parameters, and in yet another aspect, the output of the model is used to determine an insulin sensitivity factor, or in other aspects, the output of the model is used to determine an insulin-to-carbohydrate ratio, to determine a total daily dosage of insulin to cover a patient's basal insulin needs, or to determine an indicator of gastric emptying.

In yet another aspect, the analyzing step includes using various parameter estimation techniques, such as, for example, wherein at least one of the various parameter estimation technique is a technique selected from the group consisting of expectation maximization, maximum likelihood estimation, extended Kalman Filtering, extended Kalman smoothing, unscented Kalman filtering, unscented Kalman smoothing, and unscented Rauch-Tung-Striebel smoothing.

In still another aspect, the present invention includes a system for controlling insulin delivery to a patient, comprising: a glucose monitor for providing glucose level data representative of an amount of glucose in a patient's blood stream; an input device for inputting carbohydrate intake data; a processor configured to receive the glucose level data and carbohydrate intake data, the processor programmed to analyze the received glucose level and carbohydrate intake data using a model to predict a future glucose level of the patient, and to provide insulin and carbohydrate intake recommendations based on the predicted future glucose level.

In another aspect, the system further comprises an insulin pump in operable communication with the processor, and wherein the insulin recommendations are commands transmitted by the processor to the insulin pump to control the pump to deliver insulin to the patient in accordance with the insulin recommendations. In still another aspect, the model is an extended Bergman Minimal Model.

In yet another aspect, the system further comprises a memory in operable communication the processor in which glucose level, carbohydrate intake data, predicted glucose level data and recommendations are stored.

In a further aspect, the present invention includes a system for predicting the future glucose level of a patient based upon patient specific parameters, such as glucose level history, insulin delivery history, carbohydrate intake and exercise history, comprising: an input device for inputting values of at least one parameter selected from the group consisting of glucose level, carbohydrate intake, insulin type, insulin delivery amount, and exercise intensity and duration; a memory for storing values related to glucose level history, insulin delivery history, carbohydrate intake and exercise, including inputted values for the at least one parameter selected from the group consisting of glucose level, carbohydrate intake, insulin type, insulin delivery amount, and exercise intensity and duration; and a processor in operable communication with the input device and the memory, the processor programmed retrieve data from the memory to calculate patient specific parameters related to the prediction of a future glucose level of the patent, the processor also programmed to use the calculated patient specific parameters as inputs to a model employing algorithms to produce an output related to a future glucose level of the patent, the processor also programmed to uses rule sets and assumptions to simplify production of the output, and wherein the processor is programmed to transform the retrieved data by weighting the data to improve a quality of the output of the model.

These and other advantages of the invention will become apparent from the following more detailed description when taken in conjunction with the accompanying drawings of illustrative embodiments.

### BRIEF DESCRIPTION OF THE DRAWINGS

FIG. 1 is a schematic diagram illustrating an exemplary embodiment of a controller and its various components in operable communication with one or more medical devices, such as a glucose monitor/meter or drug delivery pump, and optionally, in operable communication with a remote controller device.

FIG. 2 is chart illustrating data taken from a Continuous Glucose Monitoring system and is presented along with data representing the glucose output generated from a best fit model, and difference between the two. The chart includes lines developed using CGM data, ordinary differential equations, the corresponding ARD of those two lines and discrete glucose measurements labeled as SMBG.

FIG. 3 is a chart showing blood glucose level as a function of time during a representative day for a patient. This chart shows data taken from a continuous glucose monitoring system along with data predicted using a model using ordinary differential equations such as set forth in the specification below as well as specific points labeled SMBG. This graph uses the parameters modified using the system identification process applied to this patient's insulin doses in this episode to improve control of the patient's blood glucose. As a result, the patient spends an additional 15 hours within a desired target range of blood glucose.

FIG. 4 is a flow chart illustrating an embodiment of the present invention for providing therapy recommendations to a patient.

FIG. 5 is a graph representing the patient's insulin sensitivity factor as a function of insulin dose.

FIG. 6 is a flow chart illustrating one method of providing insulin therapy recommendations to a patient based on a physiological model embodying principles of the present invention.

FIG. 7 is a flow chart illustrating another embodiment of the present invention wherein a physiological model is selected, then simplified using appropriate simplifications, to reduce computational complexity.

FIG. 8 is a flow chart illustrating another embodiment of the present invention utilizing decomposition of meal, insulin and other events to simplify a model used to provide therapy recommendations to a patient.

FIG. 9 is a graph illustrating the effect of decomposition as applied to meal events on the glucose level of a patient.

FIG. 10 is a graph illustrating the effect of decomposition as applied to insulin administration events on the insulin level in a patient's blood stream.

FIG. 11 is a chart showing blood glucose level as a function of time during a representative day for a patient. This chart shows data taken from a continuous glucose monitoring system along with data predicted using a model using ordinary differential equations in accordance with one embodiment of the present invention.

FIG. 12 is a chart showing the plasma insulin level of the patient of FIG. 5 as a function of time similar to the chart of FIG. 11.

FIG. 13 is a graph illustrating the amount of carbohydrates in the gut of the patient of FIG. 11 as a function of time.

FIG. 14 is a chart showing blood glucose level as a function of time during a representative day for a patient. This chart shows data taken from a continuous glucose monitoring system along with data predicted using a model using ordinary differential equations in accordance with one embodiment of the presenting invention.

FIG. 15 is a chart showing the blood glucose data of FIG. 14 plotted against data estimated by the model showing that the model fit is improved by using improved of the model parameters.

FIG. 16 is a flow chart illustrating an embodiment similar to that of FIG. 8, but including applying weighting to model parameters to improve the fitment of the model.

FIG. 17 is a graph illustrating weighting of an insulin event in accordance with one embodiment of the present invention.

FIG. 18 is a graph illustrating weighting of the use of long acting insulin in accordance with one embodiment of the present invention.

FIG. 19 is a graph illustrating weighting of the type, amount and timing of a meal in accordance with one embodiment of the present invention.

FIG. 20 is a chart showing blood glucose level as a function of time during a representative day for a patient. This chart shows data taken from a continuous glucose monitoring system along with data predicted using a model composed of ordinary differential equations in accordance with one embodiment of the present invention.

FIG. 21 is a chart similar to that of FIG. 20, except that the model has now incorporated temporal shifting of the meal and insulin events, resulting in marked improvement of the fit of the model data to the actual data.

FIG. 22 is a flow chart illustrating another embodiment of the present invention employing time shifting of input date to provide improved fitment of the model to the data.

### DETAILED DESCRIPTION OF THE PREFERRED EMBODIMENTS

For the purposes of promoting an understanding of the principles of the invention, reference will now be made to a number of illustrative embodiments shown in the attached drawings and specific language will be used to describe the same. It will be understood that throughout this document, the terms “user” and “patient” are used interchangeably.

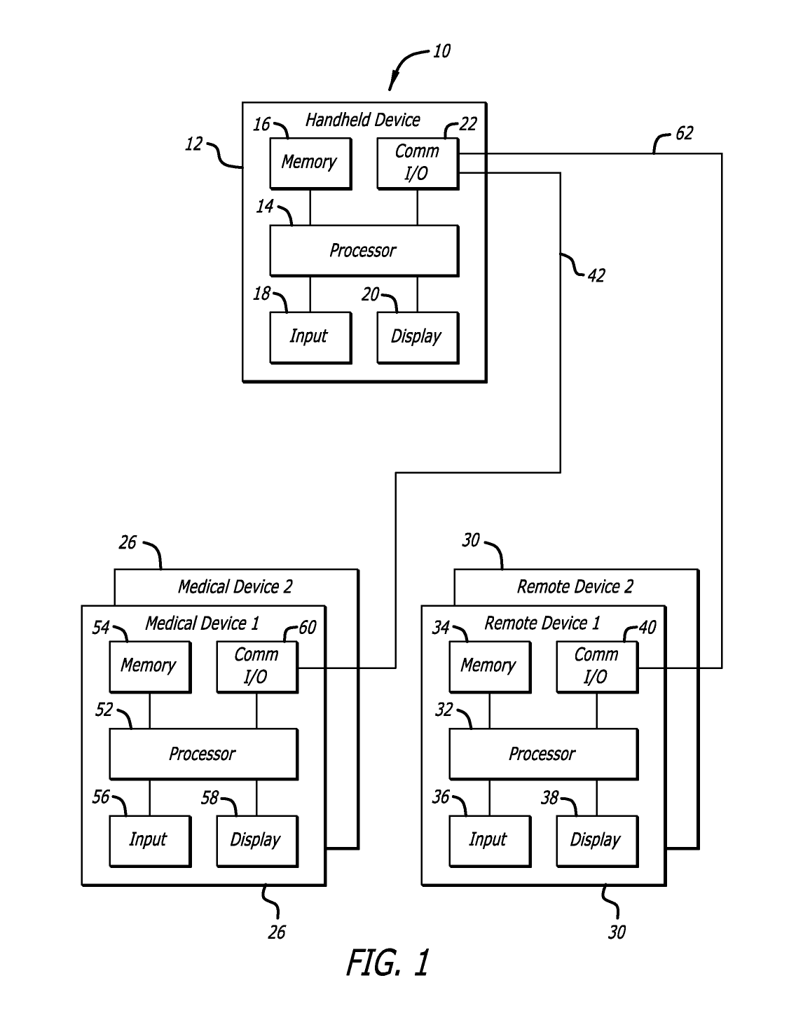

Referring now to FIG. 1, a block diagram of one illustrative embodiment of a system 10 for determining drug administration information is shown. In the illustrated embodiment, the system 10 includes an electronic device 12, which may be handheld, having a processor 14 in data communication with a memory unit 16, an input device 18, a display 20, and a communication input/output unit 24. The electronic device 12 may be provided in the form of a general purpose computer, central server, personal computer (PC), lap top or notebook computer, personal data assistant (PDA) or other hand-held device, external infusion pump, blood glucose meter, analyte sensing system, or the like. The electronic device 12 may be configured to operate in accordance with one or more conventional operating systems including for example, but not limited to the Windows® operating system (distributed by Microsoft Corporation), the Linux operating system, the Mac OS® (distributed by Apple, Inc.) and embedded operating systems such as the QNX® operating system (distributed by QNX Software Systems), the eCOS® operating system (distributed by eCosCentric Limited), Windows CE® (distributed by Microsoft Corporation) and the Palm® operating system (distributed by Palm Inc.), and may be configured to process data according to one or more conventional internet protocols for example, but not limited to, NetBios, TCP/IP and AppleTalk® (Apple, Inc.). In any case, the electronic device 12 forms part of a fully closed-loop, semi closed-loop, or open loop diabetes control system. The processor 14 is microprocessor-based, although the processor 14 may alternatively comprise one or more general purpose and/or application specific circuits and operable as described hereinafter. The memory unit 16 includes sufficient capacity to store data, one or more software algorithms executable by the processor 14 and other data. The memory unit 16 may include one or more conventional memory or other data storage devices. Electronic device 12 may also include an integrated blood glucose meter for use in calibrating a continuous glucose monitor (CGM) or for calculating insulin amounts for bolus delivery.

The input device 18 may be used in a conventional manner to input and/or modify data. The display 20 is also included for viewing information relating to operation of the device 12 and/or system 10. Such a display may be a conventional display device including for example, but not limited to, a light emitting diode (LED) display, a liquid crystal display (LCD), a cathode ray tube (CRT) display, or the like. Alternatively or additionally, the display 20 may be or include an audible display configured to communicate information to a user, another person, or another electronic system having audio recognition capabilities via one or more coded patterns, vibrations, synthesized voice responses, or the like. Alternatively or additionally, the display 20 may be or include one or more tactile indicators configured to display tactile information that may be discerned by the user or another person.

The input device 18 may be or include a conventional keyboard or keypad for entering alphanumeric data into the processor 14. Such a keyboard or keypad may include one or more keys or buttons configured with one or more tactile indicators to allow users with poor eyesight to find and select an appropriate one or more of the keys, and/or to allow users to find and select an appropriate one or more of the keys in poor lighting conditions. Alternatively or additionally, the input device 18 may be or include a conventional mouse or other conventional point and click device for selecting information presented on the display 20. Alternatively or additionally, the input device 18 may include the display 20 configured as a graphical user interface (GUI). In this embodiment, the display 20 may include one or more selectable inputs that a user may select by touching an appropriate portion of the display 20 using an appropriate implement.

Alternatively, the input device 18 may also include a number of switches or buttons that may be activated by a user to select corresponding operational features of the device 12 and/or system 10. Input device 18 may also be or include voice-activated circuitry responsive to voice commands to provide corresponding input data to the processor 14. In any case, the input device 18 and/or display 20 may be included with or separate from the electronic device 12.

System 10 may also include a number of medical devices which carry out various functions, for example, but not limited to, monitoring, sensing, diagnostic, communication and treatment functions. In such embodiments, any of the one or more of the medical devices may be implanted within the user's body, coupled externally to the user's body (such as an infusion pump, for example), or separate from the user's body. Alternatively or additionally, one or more of the medical devices may be mounted to and/or form part of the electronic device 12. Typically, the medical devices are each configured to communicate wirelessly with the communication I/O unit 24 of the electronic device 12 via one of a corresponding number of wireless communication links.

The wireless communications between the various components of the system 10 may be one-way or two-way. The form of wireless communication used may include, but is not limited to, radio frequency (RF) communication, infrared (IR) communication, Wi-Fi, RFID (inductive coupling) communication, acoustic communication, capacitive signaling (through a conductive body), galvanic signaling (through a conductive body), or the like. In any such case, the electronic device 12 and each of the medical devices include conventional circuitry for conducting such wireless communications circuit. Alternatively, one or more of the medical devices may be configured to communicate with the electronic device 12 via one or more conventional serial or parallel configured hardwire connections therebetween.

Each of the one or more medical devices 26 may include at least one processing unit 52, input/output circuitry, and/or devices 56, 58 communication ports 60 and one or more suitable data and/or program storage devices 58. It will be understood that not all medical devices 26 will have the same componentry, but rather will only have the components necessary to carry out the designed function of the medical device. For example, in one embodiment, a medical device 26 may be capable of integration with electronic device 12 and remote device 30. In another embodiment, the medical device may also be capable of stand-alone operation, should communication with electronic device 12 or remote device 30 be interrupted. In another embodiment, medical device 26 may include processor, memory, and communication capability, but does not have a display 58 or input 56. In still another embodiment, the medical device 26 may include an input 56, but lack a display 58.

In some embodiments, the system 10 may alternatively or additionally include a remote device 30. The remote device 30 may include a processor 32, which may be identical or similar to the processor 14, a memory or other data storage unit 34, an input device 36, which may be or include any one or more of the input devices described hereinabove with respect to the input device 18, a display unit 38, which may be or include any one or more of the display units described hereinabove with respect to the display unit 20, and communication I/O circuitry 40. The remote device 30 may be configured to communicate with the electronic device 12 or medical devices(s) 26 via any wired or wireless communication interface 42, which may be or include any of the communication interfaces or links described hereinabove. Although not shown, remote device 30 may also be configured to communicate directly with one or more medical devices 26, instead of communicating with the medical device through electronic device 12.

The system 10 illustrated in FIG. 1 is, or forms part of, a fully closed-loop, semi closed-loop, or open loop diabetes control arrangement. In this regard, the system 10 requires user input of some amount of information from which the system 10 determines, at least in part, insulin bolus administration information. Such insulin bolus administration information may be or include, for example, insulin bolus quantity or quantities, bolus type, insulin bolus delivery time, times or intervals (for example, single delivery, multiple discrete deliveries, continuous delivery), and the like. Examples of user supplied information may be, for example but not limited to, user blood glucose concentration, information relating to a meal or snack that has been ingested, is being ingested, or is to be ingested sometime in the future, user exercise information, user stress information, user illness information, information relating to the user's menstrual cycle, and the like. In any case, the system 10 includes a delivery mechanism for delivering controlled amounts of a drug; such as, for example, insulin, glucagon, incretin, or the like, and/or offering an alternatively actionable therapy recommendation to the user via the display 20, such as, for example, ingesting carbohydrates, exercising, and the like.

The system 10 may be provided in any of a variety of configurations, and examples of some such configurations will now be described. It will be understood, however, that the following examples are provided merely for illustrative purposes, and should not be considered limiting in any way. Those skilled in the art may recognize other possible implementations of a fully closed-loop, semi closed-loop, or open loop diabetes control arrangement, and any such other implementations are contemplated by this disclosure.

In a first exemplary embodiment of the system 10, the electronic device 12 is provided in the form of an insulin pump configured to be worn externally to the user's body and also configured to controllably deliver insulin to the user's body. In this embodiment, the medical devices may include one or more implanted sensors for providing information relating to the physiological condition of the user. Examples of such implanted sensors may include, but should not be limited to, a glucose sensor, a body temperature sensor, a blood pressure sensor, a heart rate sensor, one or more bio-markers configured to capture one or more physiological states of the body, such as, for example, HBAlC, or the like.

In those embodiments that include an implanted glucose sensor, the system 10 may be a fully closed-loop system operable to automatically monitor blood glucose and deliver insulin, as appropriate, to maintain blood glucose at desired levels. The various medical devices may alternatively or additionally include one or more sensors or sensing systems that are external to the user's body, or employ various sensor techniques for providing information relating to the physiological condition of the user. Examples of such sensors or sensing systems may include, but should not be limited to, a glucose strip sensor/meter, a body temperature sensor, a blood pressure sensor, a heart rate sensor, one or more bio-markers configured to capture one or more physiological states of the body, such as, for example, HBAlC, or the like.

In those embodiments that include an external glucose sensor, the system 10 may be a closed-loop, semi closed-loop, or open loop system operable to deliver insulin, as appropriate, based on glucose information provided thereto by the user. Information provided by any such sensors and/or sensor techniques such as those described above may be communicated to the system 10 using any one or more wired or wireless communication techniques. In this exemplary implementation, the remote device 30 may also be included in the form of a handheld or otherwise portable electronic device configured to communicate information to and/or from the electronic device 12.

In another exemplary embodiment of the system 10, the electronic device 12 is provided in the form of a handheld remote device, such as a PDA or other handheld device. In this embodiment, the medical devices 26 include at least one implantable or externally worn drug pump. In one alternative embodiment, an insulin pump is configured to controllably deliver insulin to the user's body. In this embodiment, the insulin pump is configured to wirelessly transmit information relating to insulin delivery to the handheld device 12. The handheld device 12 is configured to monitor insulin delivery by the pump, and may further be configured to determine and recommend insulin bolus amounts, carbohydrate intake, exercise, and the like. The system 10 may or may not be configured in this embodiment to provide for transmission of wireless information from the handheld device 12 to the insulin pump.

In another alternate embodiment, the handheld device 12 is configured to control insulin delivery to the user by determining insulin delivery commands and transmitting such commands to the insulin pump. The insulin pump, in turn, is configured to receive the insulin delivery commands from the handheld device 12, and to deliver insulin to the user according to the commands. The insulin pump, in this embodiment, may or may not further process the insulin pump commands provided by the handheld unit 12. In any case, the system 10 will typically be configured in this embodiment to provide for transmission of wireless information from the insulin pump back to the handheld device 12 to thereby allow for monitoring of pump operation. In any of the embodiments, the system 10 may further include one or more implanted and/or external sensors of the type described previously. A remote device 30 may also be included in the form of, for example, a PC, PDA, laptop or notebook computer configured to communicate information to and/or from the electronic device 12.

Those skilled in the art will recognize other possible embodiments of a fully closed-loop, semi closed-loop, or open loop diabetes control arrangement using at least some of the components of the system 10 illustrated in FIG. 1. For example, the electronic device 12 in one or more of the above embodiments may be provided in the form of a PDA, laptop, notebook or personal computer configured to communicate with one or more of the medical devices 26, at least one of which is an insulin delivery system, to monitor and/or control the delivery of insulin to the user. In yet another embodiment, the remote device 30 may be configured to communicate with the electronic device 12 and/or one or more of the medical devices 26, to control and/or monitor insulin delivery to the patient, and/or to transfer one or more software programs and/or data to the electronic device 12. The remote device 30 may reside in a caregiver's office or other remote location, and communication between the remote device and any component of the system 10 may be accomplished via an intranet, Internet (using, for example, the World-Wide-Web), cellular, telephone modem, RF, or other communication link. Any one or more internet protocols may be used in such communications. Alternatively or additionally, any mobile content delivery system; such as, for example, Wi-Fi, WiMAX, short message system (SMS), or other message scheme may be used to provide for communication between devices comprising the system 10.

Generally, the concentration of glucose in a person changes as a result of one or more external influences such as meals and exercise, and also changes resulting from various physiological mechanisms such as stress, illness, menstrual cycle and the like. In a person with diabetes, such changes can necessitate monitoring the person's blood glucose level and administering insulin or other blood glucose-altering drug, such as, for example, glucose lowering or raising drug, as needed to maintain the person's blood glucose within desired ranges. In any of the above described embodiments, the system 10 is thus configured to determine, based on some amount of patient-specific information, an appropriate amount, type and/or timing of insulin or other blood glucose-altering drug to administer in order to maintain normal blood glucose levels without causing hypoglycemia or hyperglycemia. In some embodiments, the processors of system 10 are configured using appropriate programming commands to control one or more external (such as, for example, subcutaneous, transcutaneous or transdermal) and/or implanted insulin pumps to automatically infuse or otherwise supply the appropriate amount and type of insulin to the user's body in the form of one or more insulin boluses. In other embodiments, the system 10 is configured using appropriate programming commands to display or otherwise notify the user of the appropriate amount, type, and/or timing of insulin in the form of an insulin recommendation. In such embodiments, the hardware and/or software of system 10 allows the user to accept the recommended insulin amount, type, and/or timing, or to reject it. If accepted, the system 10, in one embodiment, automatically infuses or otherwise provides the appropriate amount and type of insulin to the user's body in the form of one or more insulin boluses. If, on the other hand, the user rejects the insulin recommendation, the hardware and/or software of system 10 allows the user to override the system 10 and manually enter insulin bolus quantity, type, and/or timing. The system 10 is then configured using appropriate programming commands to automatically infuse or otherwise provide the user specified amount, type, and/or timing of insulin to the user's body in the form of one or more insulin boluses.

Alternatively, the appropriate amount and type of insulin corresponding to the insulin recommendation displayed by the system 10 may be manually injected into, or otherwise administered to, the patient's body. It will be understood, however, that the system 10 may alternatively or additionally be configured in like manner to determine, recommend, and/or deliver other types of medication to a patient.

The system 10 is operable, as just described, to determine and either recommend or administer an appropriate amount of insulin or other blood glucose lowering drug to the patient in the form of one or more insulin boluses. In determining such appropriate amounts of insulin, the system 10 requires at least some information relating to one or more external influences and/or various physiological mechanisms associated with the patient. For example, if the patient is about to ingest, is ingesting, or has recently ingested, a meal or snack, the system 10 generally requires some information relating to the meal or snack to determine an appropriate amount, type and/or timing of one or more meal compensation boluses. When a person ingests food in the form of a meal or snack, the person's body reacts by absorbing glucose from the meal or snack over time. For purposes of this document, any ingesting of food may be referred to hereinafter as a “meal,” and the term “meal” therefore encompasses traditional meals, such as, for example, breakfast, lunch and dinner, as well as intermediate snacks, drinks, and the like.

The general shape of a glucose absorption profile for any person rises following ingestion of the meal, peaks at some measurable time following the meal, and then decreases thereafter. The speed, that is, the rate from beginning to completion, of any one glucose absorption profile typically varies for a person by meal composition, by meal type or time (such as, for example, breakfast, lunch, dinner, or snack) and/or according to one or more other factors, and may also vary from day-to-day under otherwise identical meal circumstances. Generally, the information relating to such meal intake information supplied by the patient to the system 10 should contain, either explicitly or implicitly, an estimate of the carbohydrate content of the meal or snack, corresponding to the amount of carbohydrates that the patient is about to ingest, is ingesting, or has recently ingested, as well as an estimate of the speed of overall glucose absorption from the meal by the patient.

The estimate of the amount of carbohydrates that the patient is about to ingest, is ingesting, or has recently ingested, may be provided by the patient in any of various forms. Examples include, but are not limited to, a direct estimate of carbohydrate weight (for example, in units of grams or other convenient weight measure), an amount of carbohydrates relative to a reference amount (for example, dimensionless), an estimate of meal or snack size (for example, dimensionless), and an estimate of meal or snack size relative to a reference meal or snack size (for example, dimensionless). Other forms of providing for patient input of carbohydrate content of a meal or snack will occur to those skilled in the art, and any such other forms are contemplated by this disclosure.

The estimate of the speed of overall glucose absorption from the meal by the patient may likewise be provided by the patient in any of various forms. The carbohydrate input from the patient may take various forms. The amount of carbohydrate may be entered manually, or the meal could be photographed and image analyzed to determine the carbohydrate content. Alternatively, the system may be configured to use an input device such as a bar code reader to read the carbohydrate content from a package label or a recipe.

For a specified value of the expected speed of overall glucose absorption, the glucose absorption profile captures the speed of the meal taken by the patient. As another example, the speed of overall glucose absorption from the meal by the patient also includes the duration of time between ingesting of the meal by a person and the peak glucose absorption of the meal by that person, which captures the duration of the meal taken by the patient. The speed of overall glucose absorption may thus be expressed in the form of meal speed or duration. Examples of the expected speed of overall glucose absorption parameter in this case may include, but are not limited to, a compound parameter corresponding to an estimate of the meal speed or duration (for example, units of time), a compound parameter corresponding to meal speed or duration relative to a reference meal speed or duration (for example, dimensionless), or the like.

As another example of providing the estimate of the expected speed of overall glucose absorption parameter, the shape and duration of the glucose absorption profile may be mapped to the composition of the meal. Examples of the expected speed of overall glucose absorption parameter in this case may include, but are not limited to, an estimate of fat amount, protein amount and carbohydrate amount (for example, in grams) in conjunction with a carbohydrate content estimate in the form of meal size or relative meal size, an estimate of fat amount, protein amount and carbohydrate amount relative to reference fat, protein and carbohydrate amounts in conjunction with a carbohydrate content estimate in the form of meal size or relative meal size, and an estimate of a total glycemic index of the meal or snack (for example, dimensionless), wherein the term “total glycemic index” is defined for purposes of this document as a parameter that ranks meals and snacks by the speed at which the meals or snacks cause the person's blood sugar to rise. Thus, for example, a meal or snack having a low glycemic index produces a gradual rise in blood sugar whereas a meal or snack having a high glycemic index produces a fast rise in blood sugar. One exemplary measure of total glycemic index may be, but is not limited to, the ratio of carbohydrates absorbed from the meal and a reference value, such as, for example, a reference value derived from pure sugar or white bread, over a specified time period, such as, for example, two hours. Other forms of providing for user input of the expected overall speed of glucose absorption from the meal by the patient, and/or for providing for user input of the expected shape and duration of the glucose absorption profile generally will occur to those skilled in the art, and any such other forms are contemplated by this disclosure.

Generally, the concentration of glucose in a person with diabetes changes as a result of one or more external influences such as meals and/or exercise, and may also change resulting from various physiological mechanisms such as stress, menstrual cycle and/or illness. In any of the above examples, the system 10 responds to the measured glucose by determining the appropriate amount of insulin to administer in order to maintain normal blood glucose levels without causing hypoglycemia. In some embodiments, the system 10 is implemented as a discrete system with an appropriate sampling rate, which may be periodic, aperiodic or triggered, although other continuous systems or hybrid systems may alternatively be implemented as described above.

In one exemplary diabetes control system, one or more software algorithms may be embedded in the programming of the processor processors of the system, and may include, among other features and functions, a collection of rule sets which use (1) glucose information, (2) insulin delivery information, and/or (3) subject inputs such as meal intake, exercise, stress, illness and/or other physiological properties to provide therapy, and the like, to manage the user's glucose level. The rule sets are generally based on observations and clinical practices as well as mathematical models derived through or based on analysis of physiological mechanisms obtained from clinical studies.

As used herein, the term “model” means a set of algorithms embedded in computer programming that accepts one or more inputs, either directly from an input device or sensor, such as a CGM monitor, or indirectly through an input device such as a keyboard or other device, analyzes the inputted date, and also possible stored date, applying appropriate rule sets and assumptions, and outputs a forecasted variable value as function of some parameter, such as time. This definition is intended to be consistent with the meaning of the term “model” as used by those skilled in the art.

In an exemplary embodiment of the system, models of insulin pharmacokinetics and pharmacodynamics, glucose pharmacodynamics, meal absorption and exercise responses of individual patients are used to determine the timing and the amount of insulin to be delivered. A learning module may be provided to allow adjustment of the model parameters when the patient's overall performance metric degrades. For example, the learning module may include, for example, the use of adaptive algorithms or Bayesian estimates. An analysis model may also be incorporated which oversees the learning module to accept or reject the results generated by the learning module. Adjustments to the results of the learning module may be achieved utilizing heuristics, rules, formulae, minimization of cost function(s) or tables such as, for example, gain scheduling.

As described above, predictive models can be programmed into the processors of the system using appropriate embedded or inputted software to predict the outcome of adding a controlled amount of insulin or other drug to a user in terms of the an expected blood glucose value. The structures and parameters of the models define the anticipated behavior.

Any of a variety of conventional controller design methodologies, such as PID systems, full state feedback systems with state estimators, output feedback systems, LQG (Linear-Quadratic-Gaussian) controllers, LQR (Linear-Quadratic-Regulator) controllers, eigenvalue/eigenstructure controller systems, and the like, could be used to design algorithms to perform physiological control. They typically function by using information derived from physiological measurements and/or user inputs to determine the appropriate control action to use. While the simpler forms of such controllers use fixed parameters (and therefore rules) for computing the magnitude of control action, the parameters in more sophisticated forms of such controllers may use one or more dynamic parameters. The one or more dynamic parameters could, for example, take the form of one or more continuously or discretely adjustable gain values. Specific rules for adjusting such gains could, for example, be defined either on an individual basis or on the basis of a patient population, and in either case will typically be derived according to one or more mathematical models. Such gains are typically scheduled according to one or more rule sets designed to cover the expected operating ranges in which operation is typically nonlinear and variable, thereby reducing sources of error.

Model based control systems, such as those utilizing model predictive control algorithms, can be constructed as a black box wherein equations and parameters have no strict analogs in physiology. Rather, such models may instead be representations that are adequate for the purpose of physiological control. The parameters are typically determined from measurements of physiological parameters such as blood glucose, insulin concentration, and the like, and from physiological inputs such as food intake, alcohol intake, insulin doses, and the like, and also from physiological states such as stress level, exercise intensity and duration, menstrual cycle phase, and the like. These models are used to estimate current glucose or to predict future glucose values. Such models may also take into account unused insulin remaining in the blood after a bolus is given, for example, in anticipation of a meal. Such unused insulin will be variously described as unused, remaining, or “insulin on board.”

Insulin therapy is derived by the system based on the model's ability to predict glucose for various inputs. Other conventional modeling techniques may be additionally or alternatively used including for example, but not limited to, building models from first principles.

In a system as described above, the controller is typically programmed to provide a “basal rate,” which is the rate of continuous supply of insulin by an insulin delivery device such as a pump that is used to maintain a desired blood glucose level in the bloodstream of a patient. Periodically, due to various events that affect the metabolism of a patient, such as eating a meal or engaging in exercise, a “bolus” is required. A “bolus” is a specific amount of insulin that is required to raise the blood concentration of insulin to an effective level to counteract the affects of the ingestion of carbohydrates during a meal and also takes into account the affects of exercise on the blood glucose level.

As described above, an analyte monitor may be used to continuously monitor the glucose levels in a user. The controller is programmed with appropriate software and uses models as described above to predict the affect of carbohydrate ingestion and exercise, among other factors on the predicted level of blood glucose. Such a model must also take into account the amount of insulin remaining in the blood stream from a previous bolus or basal rate infusion when determining what or whether or not to provide a bolus of insulin.

Typically, models used to calculate insulin dosage for an insulin therapy regime are specified using three numbers: an insulin sensitivity factor, insulin-to-carbohydrate ratio, and a daily dosage of insulin. Various heuristic rules exist for initially estimating these numbers and to improve glucose control. Physicians spend a considerable amount of effort in fine-tuning these numbers based upon their expertise and/or accepted titration protocols. For an individual patient, various tests, such as an IV glucose tolerance test, may be used to determine an insulin sensitivity factor for the patient. These factors tend to be individual to patients, and thus, must be determined for each patient to obtain the best control of a patient's blood glucose level.

One advantage of using a continuous glucose monitoring system is that frequent measurements of blood glucose are available for analysis. When such measurements are combined with information provided by the patient, such as, the amount of carbohydrates consumed in a meal and the amount of insulin taken daily or at specific intervals, a dynamic model describing the affect of food and subsequent insulin dosing on glucose levels can be determined. These parameters can then be used to estimate an insulin sensitivity, insulin-to-carbohydrate ratio, and total daily dosage of insulin that will produce good patient-specific glucose control.

Calculating Insulin Therapy Requirements

Various models exist that attempt to measure pancreatic responsiveness and insulin sensitivity and provide a means to evaluate their relative contributions to overall glucose tolerance. One model that has been determined by the inventors to be advantageous in performing these calculations is an extended version of the Bergman minimal model. See, e.g. Physiological Evaluation of Factors Controlling Glucose Tolerance in Man, Measurement of Insulin Sensitivity and Beta-cell Glucose Sensitivity From the Response to Intravenous Glucose, by Richard N. Bergman, Lawrence S. Philips, and Claudio Cobelli, J. Clin. Invest., The American Society for Clinical Investigation, Inc., Vol. 68, December 1981, pp. 1456-1467, the subject matter of which is hereby intended to be incorporated in its entirety. Such a model, commonly referred to as the “minimal model” derives glucose and insulin dynamics relationships using data from intravenous glucose tolerance tests. This model can be used to determine parameters of insulin responsiveness to glucose as well as for predicting a time-course of plasma insulin levels, when the glucose-time course is supplied. Additionally, an index of insulin sensitivity, commonly referred to as S_{I} is measured using a second model that predicts glucose kinetics when the insulin-time course is supplied. This model will typically supply characteristic parameters δ₁, δ₂ , and δ_{I} which represents a metabolic portrait of the glucose and insulin responsiveness of a single individual.

For example, one embodiment of the present invention uses an extended version of the Bergman minimal model with which glucose values can be calculated as follows:

Ġ=−(p₁+S_{I} X̅)G+p₁G_{b}+fk_{abs}G_{gut} Equ. 1:

d X̅/dt=p₂(I−I_{b}− X̅) Equ. 2:

İ=Ξ(t)−kₑᵢI Equ. 3:

G_{gut}=Dkₑₘₚ²└βe^{-k}^{_{abs}}^{I}−(γt+β)e^{-k}^{ₑₘₚ}^{t}┘ Equ4:

where:

G_{b}=fasting plasma glucose concentration in absence of insulin O(100 mg/dl).

G=blood glucose concentration

p₁=rate of insulin independent glucose clearance O(10⁻² min⁻¹).

S_{I}=insulin effectiveness O(10⁻⁴ L/min-mU).

f=lumped glucose distribution volume and fractional gut absorption O(10⁻²L⁻¹).

X̅=effective insulin concentration.

p₂=rate of appearance/disappearance of active insulin O(10⁻² min⁻¹).

I=insulin concentration O(10⁻² U/L).

I_{b}=basal insulin concentration O(10⁻² U/L).

Ξ=rate of insulin absorption from the subcutaneous injection/administration site.

k_{abs}=rate of carbohydrate absorption for the gut O(10⁻¹ min⁻¹).

kₑᵢ=rate of insulin clearance O(10⁻² min⁻¹).

G_{gut}=mass of carbohydrate in the gut.

D=mass of carbohydrates in a given meal (grams)

kₑₘₚ=rate of gastric emptying O(10⁻² min⁻¹).

$$β = \frac{1}{{k}_{emp} - {k}_{abs}}$$

γ=β² and

t=time.

By using various parameter estimation techniques known to those skilled in the art, all unknown parameters in the model can be estimated. For example, parameter estimation may be performed on a digital computer using known linear least squares processes programmed into the computer. The accuracy of the parameter estimate may, for example, be evaluated using a Fisher Information Matrix. Finally, analysis of the relation between estimated parameters within groups may be performed, for example, using a student's t-test and regression analysis. Those skilled in the art will immediately understand that other statistical methods may be used to analyze and model the data measured by the CGM to determine the various parameters needed to predict present and/or future plasma glucose levels.

By invoking certain assumptions, such as, for example, a pseudo-steady status assumption about the user's blood glucose level status, the above identified embodiment of the model can be used to determine the change in blood glucose following a bolus of insulin can be determined accordingly:

$$\begin{matrix}0 = - ( {p}_{1} + {S}_{1} \overline{X} ) G + {p}_{1} {G}_{b} & Equ . 5 \\ 0 = {p}_{2} ( I - {I}_{b} - \overline{X} ) & Equ . 6 \\ G = \frac{{p}_{1} {G}_{b}}{( {p}_{1} + \frac{{S}_{1} {I}_{p}}{{V}_{i}} )} & Equ . 7 \\ Δ G = - {G}_{b} ( \frac{{S}_{1} {I}_{p} / {V}_{i} {p}_{1}}{1 + {S}_{1} {I}_{p} / {V}_{i} {p}_{1}} ) & Equ . 8\end{matrix}$$

where:

ΔG=change in plasma (blood) glucose level.

Iₚ=increase in insulin concentration following a bolus.

Vᵢ=distribution volume of insulin in the body.

This method allows the calculation of the amount of insulin needed to make a blood glucose correction. For example, to determine prandial insulin requirements of a patient, the rise in blood glucose resulting from a meal consumed by the patient can be estimated as follows:

$$\begin{matrix}Δ {G}_{meal} = {\int }_{0}^{∞} {fk}_{abs} {G}_{gut} d t & Equ . 9 \\ {G}_{gut} = D ( {βe}^{- {k}_{abs} t} - [ γ t + β ] {e}^{- {k}_{emp} t} ) & Equ . 10 \\ Δ {G}_{meal} = fD & Equ . 11 \\ fD = {G}_{b} ( \frac{\frac{{S}_{I} {I}_{p}}{{V}_{i} {p}_{1}}}{1 + \frac{{S}_{I} {I}_{p}}{{V}_{i} {p}_{1}}} ) & Equ . 12\end{matrix}$$

Generally, the above equations will be embodied in appropriate software programs designed to run on a general purpose, or specific purpose computer. Data received from the CGM, as well as intermediate results from calculations performed by a microprocessor in the computer, may be stored either in permanent or semi-permanent or transitory memory, such as RAM or a hard drive or other storage media. The software program operating on and controlling the computer, will iteratively solve the above-identified equations using the data provided by the CGM, intermediate calculation, or stored in memory, to provide parameter values for the variables identified.

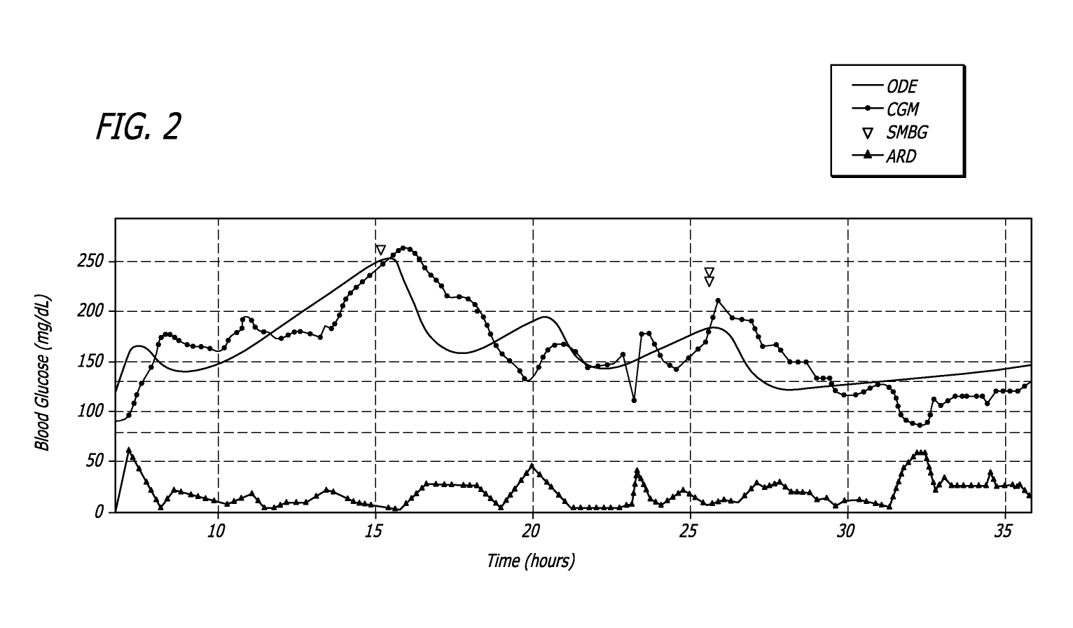

FIG. 2 is a chart illustrating data taken from a continuous glucose monitoring system and is labeled “CGM.” The chart also displays a line drawn using the output from a model employing an ordinary differential equation (ODE). The ODE data is generated by the model from the patient's blood glucose, meal, insulin, exercise and other patient information. The absolute relative difference (ARD) between the CGM results and the ODE results, calculated in a pointwise fashion, is illustrated by the line labeled ARD. Also presented are discrete blood glucose measurements taken using, for example, finger stick methods, and are labeled as SMBG. The correspondence of the CGM and ODE curves on the chart illustrates the ability of the underlying physiologic model to predict patient-specific parameters relating to the glucose and insulin metabolism.

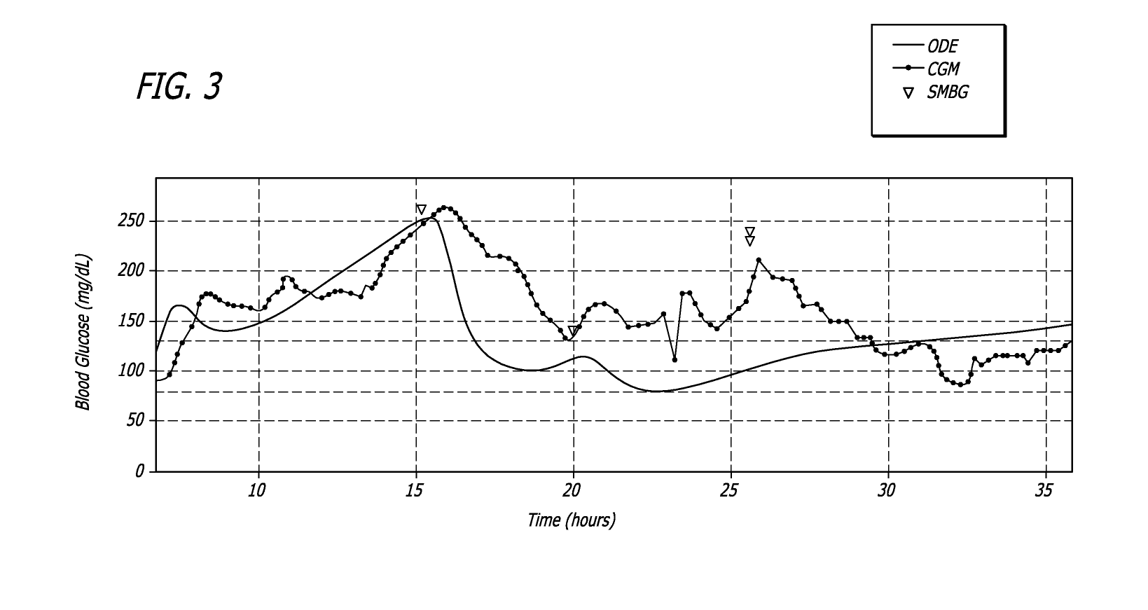

FIG. 3 is a chart showing blood glucose level as a function of time during a representative day for a patient. This chart shows data taken from a continuous glucose monitoring system along with data predicted using a model using ordinary derivative equations such as set forth in the specification above as well as available SMBG data. This graph uses the parameters modified using the system identification process applied to this patient's insulin doses in this episode to improve control of the patient's blood glucose. As a result, the patient spends an additional 15 hours within a desired target range of blood glucose. In this example, only the insulin dose can be altered using the model embodying the method described here. The patient performed an insulin dose adjustment (or delivery) around the time of the labeled SMBG data. As a result, the predicted glucose level prior to the first SMBG measurement (at around 15 hours) remains unchanged.

The parameters may be repeatedly adjusted to evaluate the selection of optimal settings for the model when viewed from a clinical perspective. For example, parameters may be adjusted to improve the amount of time a patient's glucose level is within the desired range, yet the adjusted parameters may also raise the amount of time the glucose level is at extreme levels outside the desired range. In such a case, further adjustment of the parameters is made and the model repeated until a satisfactory balance between clinical risk and benefit has been achieved.

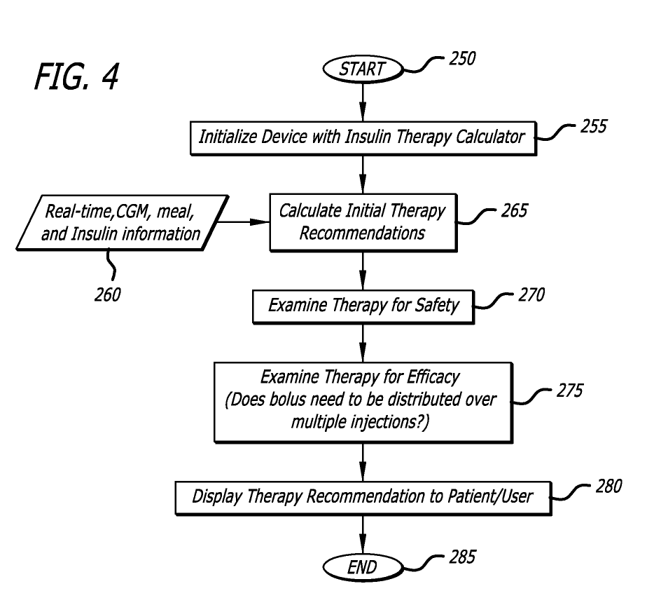

One example of a process using the principles of one embodiment of the present invention is illustrated in FIG. 4. The process starts at box 250. A programmable device designed to assist a patient in determining how much insulin he or she should administer/inject is initialized with an insulin therapy calculator in box 255. This programming may be running (through suitable software) on either a computer, or a portable device, such as a specialized device such as a handheld insulin calculator/pump controller, a PDA or other such device.

Glucose data, measured either using traditional finger stick means or using continuous glucose monitoring, meal and insulin information are retrieved in box 260 and used to calculate initial therapy recommendations 265. These calculations may be accomplished using suitable programming commands operating on a processor in the device and incorporating software commands embodying, for example, the equations and calculations set forth above.

Once the initial therapy recommendations have been calculated in box 265, they are evaluated for safety in box 270. For example, the recommendations may be evaluated to determine if the patient, administering insulin in accordance with the recommendations, risks entering a hypoglycemic state.

The therapy recommendations are then evaluated for efficacy in box 275. For example, it may become apparent that the bolus amount needs to be distributed over multiple injections or administrations to prevent hypoglycemia.

After evaluation in box 275, the recommendations are output to a display visible to the patient in box 280, or alternatively, to a pump controller, and the process ends in box 285.

The above process provides an automated means to determine appropriate insulin delivery settings based on glucose, insulin delivery, meal and other diabetes related data. Presently, insulin delivery settings are determined by various heuristic rules that require physicians to spend a considerable amount of effort in fine-tuning these numbers based upon their expertise and/or accepted titration protocols. For an individual patient, various tests, such as an IV glucose tolerance test, may be used to determine an insulin sensitivity factor for the patient. These factors tend to be individual to patients, and thus, must be determined for each patient to obtain the best control of a patient's blood glucose level. Moreover, using a calculator in accordance with the embodiments described herein allows insulin delivery to be spread over multiple doses, which may improve the efficiency of the insulin delivery, as a single large bolus of insulin may not be fully metabolized, and thus not fully effective in metabolizing glucose in the patient.

Distributed Bolus Calculator

The Bergman model described above describes the physiological glucose response to insulin in meals and suggests that the insulin sensitivity decreases for boluses about some patient specific value. For example, up to some bolus amount, such as 5 units for a particular person, the insulin effect will be maximal, but above this amount the effect will be reduced. This reduction in insulin affects results in lower overall effectiveness of the insulin and essentially wastes insulin. Up until now, the magnitude of an insulin dose has been ignored when determining insulin therapy. Research, however, suggests that large plasma insulin concentrations contribute to insulin resistance and resulting obesity which may exacerbate or accelerate complications due to diabetes.

By taking into account the size of an insulin dose in a multi-objective optimization problem, insulin therapies can be designed to minimize the combined risks of hyperglycemia and hyperinsulemia which may produce better patient outcomes. The above-described model can recognize the importance of controlling the size of an individual insulin bolus so that the effectiveness of the insulin is maximized. Utilizing the extended Bergman models set forth above, a bolus calculator is embodied in software designed to run on the microprocessor of a computer or other device to ensure that a therapy regime can be determined that prevents a bolus of insulin from being delivered that exceeds a calculated effectiveness for the patient. The calculator embodied in the software can effectively utilize glucose measurements from any source, such as continuous glucose data from a CGM. This is advantageous over traditional bolus calculators that only use present or nearly present glucose data.

This model allows a bolus to be distributed in time in such a way as to maintain the effectiveness of the insulin in reducing glucose levels and to ultimately save the patient money on insulin as well as preventing any unwanted effects, such as a reduction in insulin sensitivity with concomitant loss in control of glucose level. Using the calculator such as described is also safer than traditional methods as large boluses of insulin are avoided and since insulin is delivered over a longer period of time, there is more opportunity to interrupt insulin delivery if it becomes clear that too much insulin was recommended. The bolus calculator of one embodiment of the present invention can be applied to a variety of insulin delivery methods, such as an insulin pump, injection therapy with fast acting insulin, injection therapy with mixed action insulin, oral medication, and various combinations of these therapies.

Using an insulin pump, for example, the calculator, in one embodiment, may be used to determine an extended bolus or a dual bolus of insulin. Software is provided to a microprocessor to allow formulation of a multi-objective optimization problem where the decision variables are insulin dose and blood glucose. This allows one to take advantage of the increasing marginal effectiveness of small insulin boluses by allowing a compromise between blood glucose target and insulin dose.

Typically, as described above, patients calculate the amount of insulin necessary to cover an expected quantity of carbohydrates consumed in a meal. The model can be programmed to determine a bolus amount where insulin effectiveness in the patient becomes reduced. While this parameter is considered a constant, different values may be estimated for different doses of insulin. The point at which the insulin effectiveness is less than maximal will be referred hereafter as the effectiveness limit (EL).

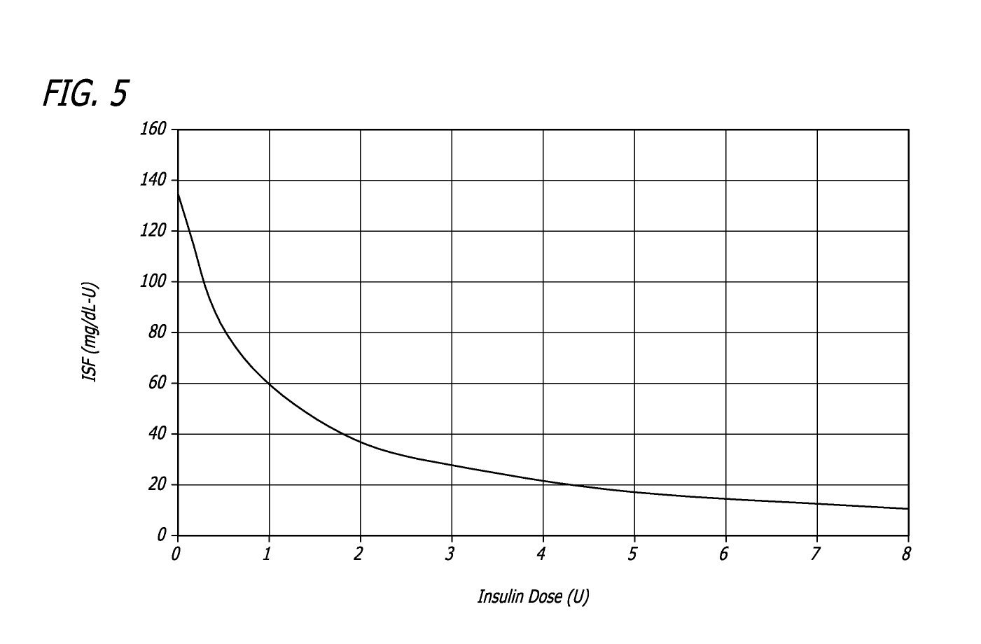

FIG. 5 provides a plot illustrating the decreasing marginal effectiveness of each unit of insulin for a patient. In such a case, when a bolus is recommended that exceeds the EL, it is divided into one or more boluses to be delivered at different times. The subsequent deliveries occur after some point where the plasma insulin levels have dropped to a point where the subsequent delivery would have full or close to full effectiveness. The pump control device may deliver these boluses automatically or with a confirmation prompt or the system could inform the user to deliver these boluses. Alternatively, the calculator may simply provide the user with this information and let the user fully control how the insulin is delivered.

In another embodiment, the calculator is used to develop a therapy regime where insulin is delivered at a reduced rate for maintenance of maximal insulin effect at a concentration consistent with the effectiveness level limit. While this appears to be much like using an extended bolus, the calculator may also determine, in addition to the amount of insulin to be delivered, the optimal rate of delivery which is defined as delivering insulin as fast as possible, but slow enough to ensure maximal effectiveness is maintained. Thus the calculator is provided with a mathematical model embedded in appropriate software programming that controls a processor in a device such that the calculations of this embodiment of the model forecasts the appearance of insulin in the plasma as a result of subcutaneous infusion and the previously mentioned multi-objective optimization. Additionally, the calculator may also calculate delivery of insulin at a variable rate rather than at just one or more constant rates.

In another embodiment, where insulin is delivered by injection, the bolus may be calculated and broken up into multiple boluses. Additionally, the calculator may include functionality embodied in the software that provides for mixing long-acting and short-acting insulin to mitigate the effects of reduced insulin effectiveness. Thus, the calculator may analyze a patient's glucose-insulin-meal data and recommend an appropriate mix of long-acting and short-acting insulin to maximize the insulin's effectiveness and perhaps reduce the frequency of multiple injections.

If the patient's glucose level indicates that a correction is necessary and the correction is undertaken, and the patient has been monitoring their glucose either naturally with CGM or by discrete monitoring, the bolus calculator may be used to determine if too much insulin is being given. Bolus calculators traditionally have an input called “insulin onboard” (IOB) that is taken into account in the calculation, and another embodiment of the bolus calculator described above has a similar functionality.

IOB may be calculated based upon previous insulin delivered and consumption of the insulin. The parameters describing the consumption of the insulin may be calculated using the software embodied in the present invention. The calculator described above may also take into account insulin planned for delivery, that is, future delivery of insulin as described above in relation to providing a continuous or almost continuous delivery of insulin to maintain the level of insulin in the blood at the most effective level. For example, if the calculator determines that another three units of insulin is needed to cover all of the carbohydrates consumed, but that eight units of insulin are already planned for delivery, either delivered at a slow rate or planned for another bolus, the calculator may recommend a change to either continue at the slow rate for less time or change the planned delivery of a large bolus to a smaller size bolus. Alternatively, if the calculator determines that no insulin is needed or that too much insulin has already been delivered, the calculator may recommend cancelling any further insulin delivery and warning the user that hypoglycemia may occur.

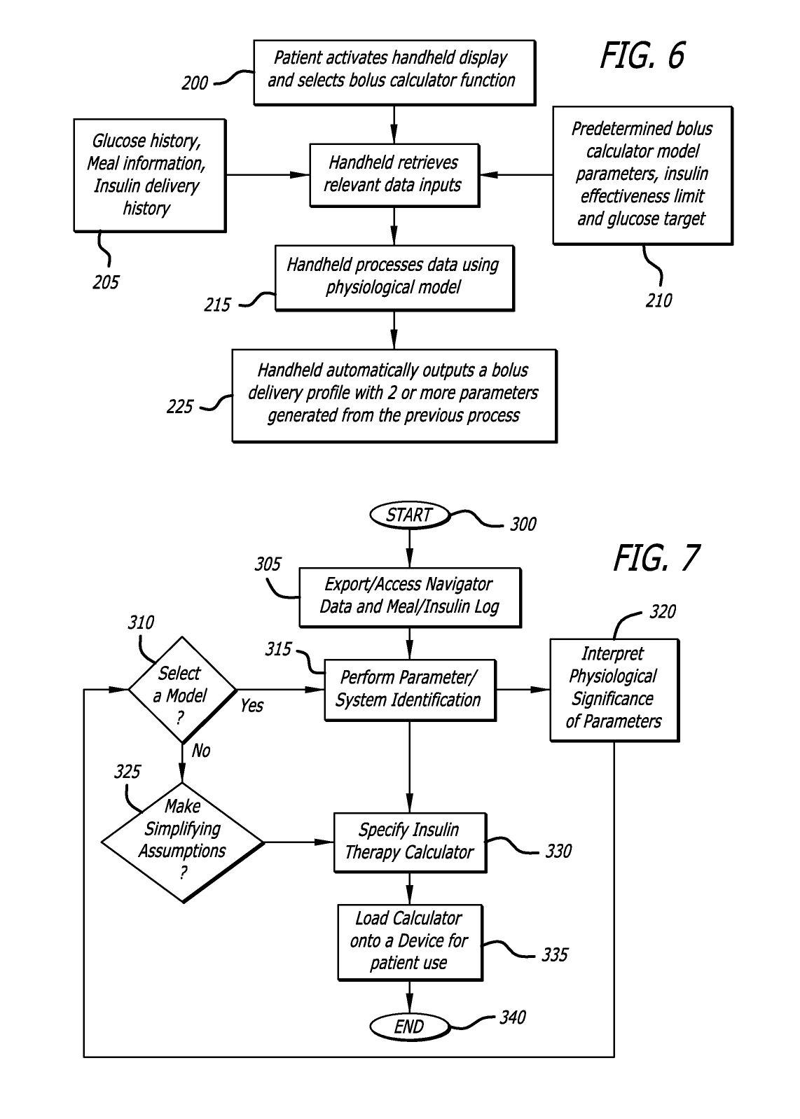

FIG. 6 illustrates one example of a process incorporating principles of the above described invention in a bolus calculator. Such a calculator includes the ability to compensate for reduced insulin effectiveness. It also includes a safety feature in that while a patient may have the value for their insulin sensitivity set high in the calculator to allow for large doses of insulin, many small doses of insulin may lead to over dosing, with a risk that the patient may become hypoglycemic.

In box 200 of FIG. 6, a patient activates a handheld device that has a display wherein the patient can select a bolus calculator function. Those skilled in the art will recognize that this process may also be carried out on a computer, PDA, or other static or portable device having suitable programming and processing ability.

In box 215, the device retrieves relevant data inputs, such as, for example, glucose history, meal and information insulin delivery from box 205 and predetermined bolus calculator model parameters, insulin effectiveness limit and glucose target level.

In box 220, the device process the data using a physiological model, such as the model set forth herein. Once processing is completed, the device outputs a bolus delivery profile with two or more parameters generated during the process of box 220. The bolus profile may be a single bolus event, or it may include for multiple smaller boluses. The output may be a command or a series of commands to an insulin pump to program the pump to deliver insulin in accordance with the outputted profile. Alternatively, the bolus delivery profile may include commands to an insulin pump to provide for an extend bolus with a recommended rate and duration of the insulin delivery. The profile may also include other commands, such as start times, times of delivery, or delays to be included to defer starting of the pump until a selected time in the future.

Alternatively, the output may be a bolus profile to be used when manually injecting insulin. All of the other parameters listed above would apply to this embodiment also.

Further, the output may include commands to delay displaying a recommendation for insulin delivery to the patient. The duration of the delay maybe predetermined, or may be calculated based upon the results of the modeling process.

In another embodiment, the output may be a series a series of recommended two or more delivery amounts and corresponding elapsed times when to notify and display the recommended amount to the patient, where the maximum number of notifications is predetermined. In yet another embodiment, the model may be used to update a recommendation based on new glucose measurement data, if available, or the process may prompt the patient to take a new glucose reading by, for example, initiating a finger stick reading. As will be apparent to those skilled in art, the exemplary process illustrated in FIG. 6 may also be initiated before the patient selects the bolus calculator function in box 200. This embodiment would be particularly advantageous where glucose data is being continually retrieved using a continuous glucose monitoring system.

The types of output from the process may be incorporated into the configuration of a bolus calculator utilizing the various embodiments described above. For example, a patient may pre-configure the system for how many injections to use for a given bolus or how many injections would be allowable for a given meal event.

Distilling an Insulin Sensitivity Factor and Insulin to Carbohydrate Ratio

Currently, insulin therapy is specified by an insulin sensitivity factor, an insulin to carbohydrate ratio and a total daily dose of insulin. Various heuristic rules exist for initially estimating these parameters, and physicians spend considerable effort and time fine tuning these parameters to improve glucose control.

Using an extended version of the Bergman Minimal Model, as set forth above, and using various parameter estimation techniques, all unknown parameters in the model can be estimated from data representing frequently measured blood glucose values of a patient and combining those measurements with meal and insulin delivery data using a dynamic model describing the effect of food and subsequent insulin dosing on plasma glucose levels. These parameters can them be used to estimate insulin sensitivity, insulin-to-carbohydrate ratio, and total daily dose of insulin that will produce good patient-specific glucose control.

For example, by invoking a pseudo-steady state assumption, one can find the change in blood glucose following the delivery of a bolus of insulin, such as is set forth in Equations 5-8 detailed above.

Using the definition of the insulin sensitivity factor, that is, the drop in blood glucose following a one unit dose of rapid-acting insulin, the insulin sensitivity factor becomes:

$$ISF = {G}_{b} ( \frac{\frac{{S}_{I}}{{V}_{i} {p}_{1}}}{1 + \frac{{S}_{I}}{{V}_{i} {p}_{1}}} ) \qquad Equ . 13$$

The insulin-to-carbohydrate ratio is then defined in terms of the insulin sensitivity factor and the expected rise in blood glucose resulting from a meal:

$$\begin{matrix}Δ {G}_{meal} = {\int }_{0}^{∞} {fk}_{abs} {G}_{gut} d t & Equ . 14 \\ {G}_{gut} = D ( β {e}^{- {k}_{abs} t} - [ γ t + β ] {e}^{- {k}_{emp} t} ) & Equ . 15 \\ Δ {G}_{meal} = fD & Equ . 16 \\ I \text{:} C \approx f / ISF & Equ . 17\end{matrix}$$

Where I:C=insulin to carbohydrate ratio, and f is a lumped parameter.

It should be noted that the estimate of the ISF need not be done using the proposed method. A physician specified ISF or an ISF determined using an alternative method may be used in the equation to determine the insulin to carbohydrate ratio.

It will be immediately apparent to one skilled in the art that the above model allows for the calculation of the total daily dose (TDD) of insulin needed to cover the expected number of carbohydrates that will be consumed on any given day, as well as the fraction of the total daily dose of insulin that will be used to cover basal needs. The expected number of carbohydrates consumed on any given day is typically around 200 grams, although this value may vary widely. The fraction of the total daily dose that will be used to cover daily basal needs is typically 0.4 to 0.5 units. Thus, for example:

$$TDD = \frac{{fD}_{daily}}{0.4 \times ISF} \qquad Equ . 18$$

Even if an analytical expression is not readily identifiable, repeated numerical simulations to determine the ISF can be performed. Determining the ISF in this manner, insulin to carbohydrate ratio and the total daily dose of insulin may then be calculated using the equations set forth above. Alternatively, each treatment parameter can be determined exclusively from numerical simulations.

Finally, if it is desirable to avoid parameter identification of a model, one could parse the log file of a continuous glucose monitoring system for meal and insulin events and note plasma glucose change at some time post-event. The change in plasma glucose could then be used to estimate the insulin sensitivity factor and insulin-to-carbohydrate ratio, either independently or as presented in the previous derivation. A rule similar to that presented previously could then be used to calculate the total daily dose of insulin, or equivalently the basal insulin requirements of the user. Using such a feature could result in decreased time for clinicians in calculating parameters individual to specific patients as well as a decreased error rate for patients along with improved patient outcomes.

Typically, the above equations will be embodied in appropriate software programs designed to run on a general purpose, or specific purpose computer. Data received from the CGM, as well as intermediate results from calculations performed by a microprocessor in the computer may be stored either in permanent or semi-permanent or transitory memory, such as RAM or a hard drive or other storage media. The software program will iteratively solve the above-identified equations to provide parameter values for the variables identified.

In practice, a clinician may download the log of a continuous glucose monitoring sensor and, taking the data from that log, input the data into a computer program running on a processor with associated memory. The processor manipulates the data in accordance with program commands simulating the equations set forth above, and outputs the insulin sensitivity factor, insulin-to-carbohydrate ratio and total daily dose of insulin needed. The clinician may then modify this therapy according to their objectives and/or expertise and may then provide it to the patient either in an electronic form such as a hand-held computer or PDA such as the FreeStyle Navigator® Continuous Glucose Monitoring System that is distributed by Abbott Diabetes Care, or by some other means.

Accelerated Parameter Identification By Modifying Meal-Insulin and Exogenous Input Events

The concept of using CGM data and various exogenous inputs, such as meals, insulin, and exercise, to identify important patient characteristics in order to improve insulin-dosing strategy has been shown to be both feasible and beneficial. One of the primary challenges in making such a framework a practical reality is the computational demands and complexity of the model used in the system identification model.

System identification is a common term in the in field of automatic control/systems theory where the parameters of an assumed model are bring identified using available measurements. The measurements typically cover one or more signals over a course of time. A relatively simple model requires fewer signals representative of a selected measurement parameter and/or taken over shorter duration, assuming the sampling period of the signal is sufficient. A relatively complex model requires several signals and/or longer measurement durations, and certain conditions are imposed on the signals so that they contain enough information to infer the parameter values.

A relatively higher complexity model allows for better fitment of more of the patient's specific characteristics while a relatively lower complexity model reduces the time required to perform the identification. In making the trade off, care must taken to introduce the proper amount of complexity to ensure the best fit of the model to the measurements so as to ensure that accurate prediction of future states can be obtained without incurring an unacceptable penalty in terms of computation time necessary to calculate the necessary parameter values from the data.

One of the components of the model used in the system identification process is the model of exogenous input such as meals and subcutaneous insulin injections. These inputs are typically modeled as a simple functions such as a delta function, step function, and the like, occurring at the start of the event; that is, the start of an insulin bolus convolved with the dynamic model. For example, a gastric emptying model in response to a meal input with D amount of carbohydrate content results in the rate of glucose appearance of Rₐ to be described as:

Rₐ(t)=fk_{abs}q_{gut}(t) Equ. 19:

This is the rate of gastric absorption, that is, the mass of glucose entering the blood from the intestine per unit time.

q_{gut}=D(1−e^{-(KT)}^{^{β}}) Equ. 20:

This is the mass of carbohydrate in the gut/intestine where: D=amount of carbohydrate and f, k_{abs}, β, and k are constant parameters.

See, e.g., J. D. Elashoff, T. J. Reedy, and J. H. Meyer, “Analysis of Gastric Emptying Data”, Gastroenterology, Vol. 83, pp. 1306-12, 1982, the subject matter of which is intended to be incorporated herein in its entirety. See, also, C. D. Man, M. Camilleri, and C. Cobelli, “A System Model of Oral Glucose Absorption: Validation on Gold Standard Data”, IEEE Transactions on Biomedical Engineering, 53(12), December 2006, the subject matter of which is intended to be incorporated herein in its entirety.

Other meal models make the identification process even more challenging by making the peak rate of glucose appearance a non-linear function of the amount of carbohydrates ingested. Another example is the complex modeling of insulin pharmacokinetics in relation to a subcutaneous insulin bolus (modeled as a single delta function with amplitude equal to its bolus dose). A contribution of I_{d} units of numerous rapid and long-acting insulin analogs can be modeled to affect plasma insulin I by the following model:

$$\begin{matrix}\dot{I} ( t ) = [ - {k}_{e} I ( t ) ] + [ \frac{1}{{V}_{ins}} {I}_{abs} ( t ) ] & Equ . 21 \\ {I}_{abs} = \frac{{st}^{s - 1} {T}_{50}^{s}}{{[ {T}_{50}^{s} + {t}^{s} ]}^{2}} {I}_{d} & Equ . 22\end{matrix}$$

where:

I=rate of change of the plasma insulin I,

Vᵢₙₛ=volume of plasma insulin, and

kₑ (rate of insulin clearance), Vᵢₙₛ, s, and T₅₀^{s} are constant parameters.

It should be noted that s and T₅₀^{s} are parameters that depend on the specific insulin type being used.

In the above examples, the final rows of the equations cannot be expressed in terms of fixed parameters, simply ordinary differential equations or their discrete time domain difference equations. Given that the timing of these events are taken as prior knowledge in the system identification process, it is possible to substitute specific simple input functions with several simple functions in order to greatly simplify the model dynamics. For example, the subcutaneous administration of insulin glargine has been shown to result in a trapezoidal input of insulin to the plasma. Instead of using a complex dynamic model with a single simple input function for every injection event, two delta functions could be used; the first causing the initial rise of insulin level into a steady state plateau, and the second causing the decay of the insulin level to zero. The dynamics involved can further be constrained to be linear time invariant (LTI) by a-priori non-linear transformation of the magnitude of the delta functions in order to emulate the non-linear relationship between dosing amount and the peak response amplitude.

This embodiment presents a model simplification in order to accelerate system identification time by moving away from modeling exogenous events using a combination of a simple input function for every event and a relatively complex dynamic model to a combination of several simple functions for every event and a relatively simple, linear, time and invariant dynamic model.

In the simplification process disclosed herein, the term I_{d} representing a single dose of insulin is not used in Equation 23. Rather, values for several replacement dosages are used, whose distribution of amount and spacing over time is determined by the type of insulin administered. The result from amended equation 23 is then fed directly into Equation 22 to provide the rate of change of the patient's plasma insulin over time. Using this process results in good model fit and prediction with reduced computation requirements, thus providing a solution in less time.

Previously, the trade-off between model accuracy and computational demands required the use of exogenous input models that combines a simple function representing each exogenous input event and complex series of non-linear dynamics. The non-linear dynamics place a burden on the ability to reduce computational time and hardware requirements, to the extent that implementation of a fast application (for example, less than five minutes from the start of processing to obtaining results) is almost impossible. The combination of the embodiments of the proposed methods and various other characteristics can be used to achieve this time goal. The proposed embodiments of the methods of the present invention significantly reduce the complexity of the model being identified for the treatment calculators so that the overall computational time can be minimized in order to make the system practically feasible.

One example of a process using the concepts of system identification to provide for an insulin therapy calculator is illustrated in FIG. 7. The process starts at box 300, and glucose data and meal and insulin delivery data is retrieved or accessed in box 305. This data is then used in box 315 to perform parameter and system identification in accordance with a model selected in box 310. For example, one model selected may be the model specified by the equations set forth above.

Once the process of box 315 is completed, the results may be evaluated to determine the physiological significance of the identified parameters in box 320. Depending on the results, a different model may be selected, and/or simplifying assumptions (box 325) may be made and the data re-processed. Once the results are satisfactory, an insulin therapy calculator is specified in box 330. The specified calculator is loaded onto a device or computer for patient use in box 335 and the process ends at box 340.

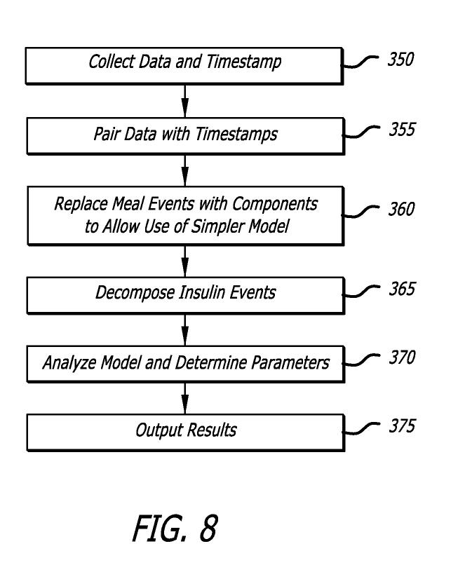

An alternative embodiment for developing and optimizing a model to predict glucose is presented in FIG. 8. In this embodiment, glucose data, meal data and insulin data, and the timestamps associated with those data, are retrieved or accessed by a processor suitably programmed using software commands in box 350.

The data is then paired with their respective timestamps in box 355. In this step, the data are paired to the nearest timestamps. For example, if continuous glucose monitoring values are typically recorded every ten minutes, and a meal is recorded at the fourth minute of the hour, then that meal is paired to the nearest 0^{th} minute of the hour of CGM data. If a meal occurs at the 9^{th} minute of the hour, then that meal is paired to the nearest 10^{th} minute of the hour of CGM data. The same process is used in pairing insulin data.

In order to simplify the computational burden of model identification, the meal events are replaced, using a process called “decomposition”, known by those skilled in the art, by components that allow the use of simpler physiological models in box 360, and the insulin events are similarly decomposed in box 365.

Without the decomposition, non-linear models are needed to model glucose appearance from meals. One example of such a non-linear model is defined by the following set of equations in response to a meal with D carbohydrates:

Rₐ(t)=∫k_{abs}q_{gut}(t) Equ. 23:

\dot{q}_{gut}(t)=└−k_{abs}q_{gut}(t)┘+Gₑₘₚₜ(t) Equ. 24:

Gₑₘₚₜ(t)=Dδ(t)βk^{β}t^{β-1}e^{-└kt┘}^{^{β}} Equ. 25:

Decomposing each of the above delta functions into one or more delta and step functions allow for a simpler model described using the following equations:

Rₐ(t)=f k_{abs}q_{gut}(t) Equ. 26:

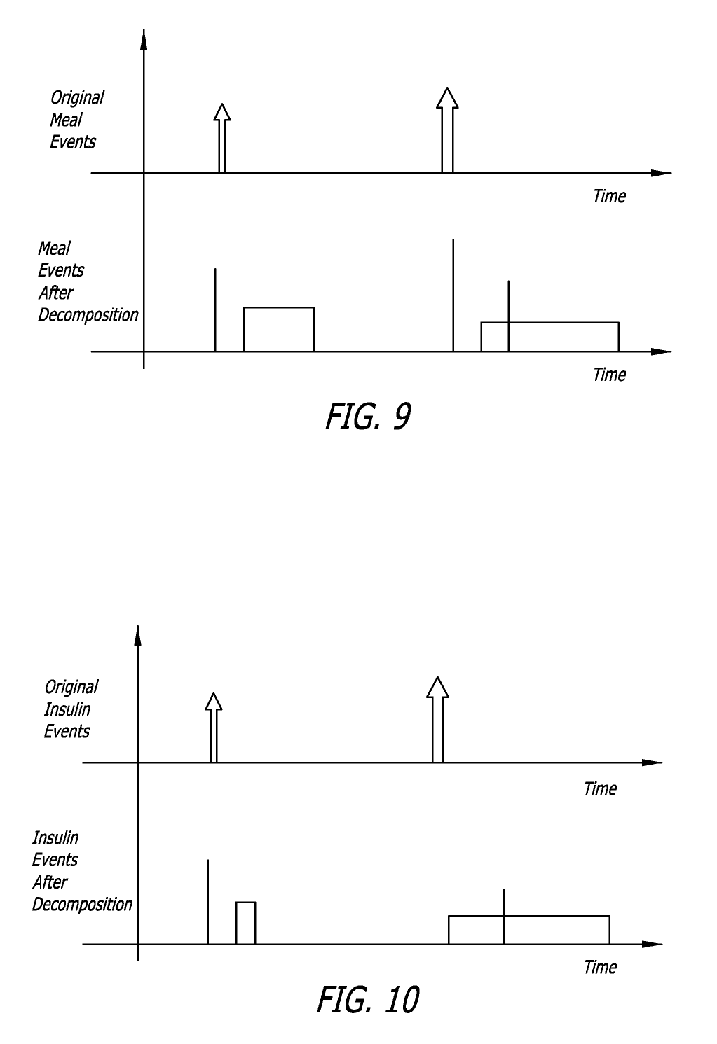

\dot{q}_{gut}(t)=└−k_{abs}q_{gut}(t)┘+d(t) Equ. 27:

where d(t) can either be a delta function or a step function of the decomposed meal events as shown in FIG. 9 below. Equation 26 is a simple linear and static equation, while equation 27 is a simple linear ODE (ordinary differential equation). Equation 25 has been rendered irrelevant by the meal event decomposition.

FIG. 9 shows an example, where two original meal events, normally modeled as delta functions at the prescribed meal times and having a magnitude proportional to the carbohydrate content, are replaced by a series of delta functions and step functions as described above in reference to the decomposition process. Since the meals are already known a-priori, there is no issue with replacing the original meal events with the new ones.

To explain further, assume that there is a meal or insulin event that occurs at some time t. The input may be treated as a delta, that is, all of the meal or insulin takes effect instantly, which will require a complicated nonlinear model to generate a useable output. Alternatively, the event may be decomposed into some number of simple inputs and these inputs can be fed into a simpler linear model. Referring again to FIG. 9, two meal events are recorded on the upper line. The relative size of the arrows indicates that these two meals are not identical. The first meal event is decomposed into a delta function (indicated by the thin line) at the time the meal is recorded, and a step function, illustrated by the rectangle, that occurs later in time and lasts for some duration. The second meal is decomposed into two delta functions, indicated by the two thin lines, and a step function, indicated by the rectangle.

By replacing the representation of the two meal events as two delta functions that serve as inputs to the meal compartment of the model with several combinations of delta and step functions as shown, the meal compartment of the model is simplified. This simplification allows use of linear time invariant meal compartment models rather than a more complex nonlinear model, thus reducing the difficulty of performing system identification and analysis. Moreover, any uncertainty in the nature of the decomposition of the meal events can be addressed by considering several possible configurations determined a-priori.

Similar to the meal events, a decomposition of the insulin events can be performed based on a-priori knowledge of the pharmacokinetics of each insulin type, which may be dose dependent. Such a decomposition is illustrated in FIG. 10, which again shows a decomposition of two insulin events into delta and step functions. Such a decomposition greatly simplifies the parameter identification and generation of output by the model.

Similar to the discussion above regarding meal events, decomposition of insulin events allow for the replacement of a combination of single delta function inputs and a nonlinear insulin compartment model with a combination of an a-priori set of multiple delta and/or step functions inputs and a simple, linear insulin compartment model. This decomposition simplifies the overall mode, which reduces calculation complexity and thus calculation time, while incurring a negligible expense due to an increased input requirement.

Without decomposition, suppose the following model is used:

$$\begin{matrix}\dot{I} ( t ) = [ - {k}_{e} I ( t ) ] + ⌊ \frac{1}{{V}_{ins}} {I}_{abs} ( t ) ⌋ & Equ . 28 \\ {I}_{abs} = \frac{{st}^{s - 1} {T}_{50}^{s}}{{[ {T}_{50}^{s} + {t}^{s} ]}^{2}} {I}_{d} & Equ . 29\end{matrix}$$

When the decomposition is applied to the above equations, only the linear ordinary differential equation (ODE) is needed to define the model:

$$\dot{I} ( t ) = [ - {k}_{e} I ( t ) ] + ⌊ \frac{1}{{V}_{ins}} {I}_{d} ( t ) ⌋ \qquad Equ . 30$$

where I_{d} is generated from the decomposed insulin events shown in FIG. 10.

Referring again to FIG. 8, once decomposition of the meal and insulin events has been accomplished, the model is analyzed and parameters are determined in box 370. Finally, the results of the process are output in box 375 for use in providing insulin therapy recommendations to the patient. Such recommendations may be used to automatically control insulin delivery to the patient. Additionally, the recommendations may be used to control a display that prompts the patient to take action to modify delivery of insulin, or may prompt the patient to ingest an amount of carbohydrate to minimize the risk that insulin already delivered may cause hypoglycemia.

Automatic Identification of an Individual's Disease Status

Using the above-identified mathematical model embedded in a processor, or processors as described above, or various embodiments thereof, glucose measurements collected as a normal part of care for a diabetic patient can be used to determine physiologically meaningful parameters for the glucose regulation of a specific individual and be used as inputs to for either automatic or manual control of the patient's insulin therapy. Those parameters include insulin pharmacokinetics/pharmacodynamics, residual beta cell function (endogenous insulin production), liver function (glycogen synthesis, fatty-acids synthesis), lipid metabolism, gastric function (for example, rate of emptying of stomach), counter-regulatory response to low blood glucose (glucagon secretion, release of glycogen stores in the liver, and gluconeogenesis), and exercise-induced glucagon secretion. Knowing the state of these functions with more accuracy and tracking them with more ease over a person's life time has the potential to improve therapy decisions for the person.

Previously, an individual's disease state had to be inferred from secondary markers from blood tests, oral glucose tolerance tests or clinical history. For example, blood tests may reveal some information about the status of the disease and residual organ/system function. One specific example is C-Peptide, measured to estimate the amount of endogenous insulin produced by the person's pancreas. The pancreas of a patient having type 1 diabetes is unable to produce insulin and therefore such a patient will usually have a decreased level of C-Peptide, whereas C-Peptide levels in type 2 diabetic patients are normal or higher than normal. However, having a more quantitative understanding of endogenous insulin production along the spectrum of health and disease has the potential to provide more accurate therapy decisions.

The method of this embodiment allows a quantified assessment of physiologically-important functions based on dynamic responses of blood glucose to the daily activities of eating, exercise and medication. Knowing these assessments can categorize a patient's disease status along the spectrum of healthy-to-diseased more accurately and rapidly.

In one embodiment, the method for determining a patient's specific parameters for disease treatment includes collecting a patient's records of blood glucose over time, insulin sensitivity function glucose, meals and medications. Data for these variables may be extracted from patient logs, or the data may also be recorded in electronic form, such as data produced by either finger stick glucose monitors, or continuous glucose monitoring systems.

Once the data has been collected, the data is entered into a database which is stored in an appropriate storage medium, such as a hard disk, thumb drive or other storage medium know by those skilled in the art. The database is then made available to a processor operating as part of a computer or other device. The processor is programmed to carry out specific functions by suitable software program commands so as to retrieve data from the database, and analyze that data in accordance with equations programmed into the software embodying the present invention. The equations forming the model of one exemplary embodiment of the present invention are set forth in Equations 1-4 described above.

Once the data has been processed by the computer, the computer fits the model either with a global optimizer or a local solver to determine the values for the various parameters solved by the equations set forth above. The output of the model is then examined, and the model parameters are transformed into physiological meaningful quantities.

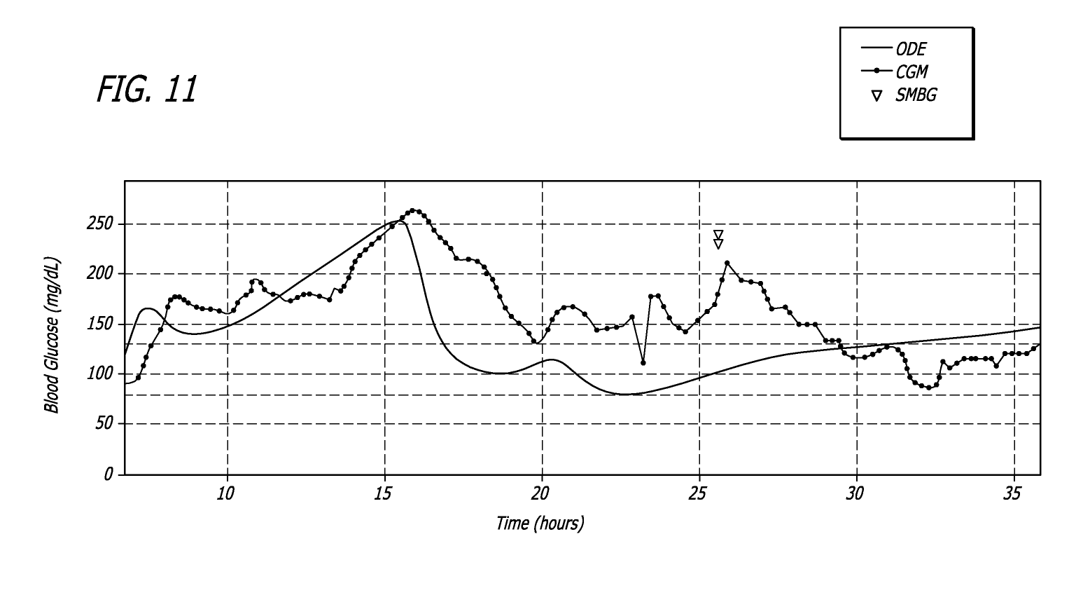

FIG. 11 is a chart showing glucose level as a function of time during a representative day for a patient. This chart shows data taken from a continuous glucose monitoring system along with data predicted using a model composed of using ordinary differential equations (ODE) such as described above. Also shown, labeled by SMBG, is a discrete blood glucose value taken using the finger stick method The predicted glucose levels represented by the line generated using the ODE model shows relatively good agreement with the actual blood glucose data measured using a CGM.

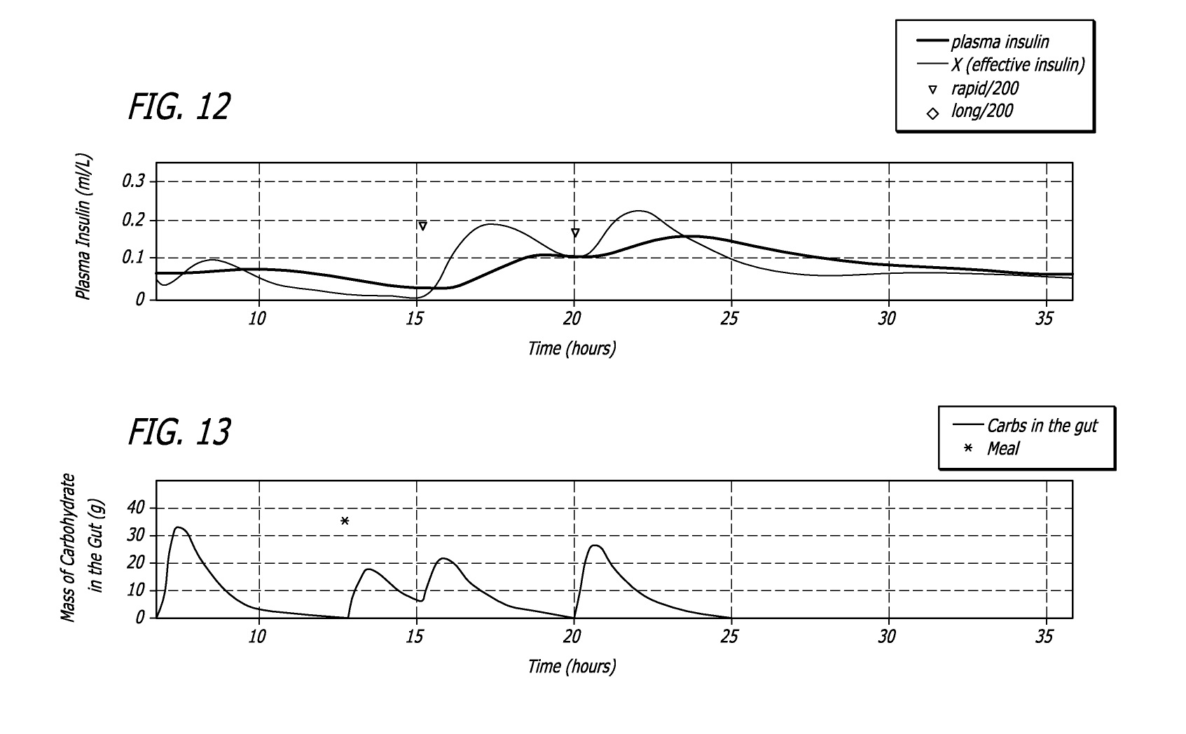

FIG. 12 is a graph showing the plasma insulin level of the patient of FIG. 11 as a function time similar to FIG. 11. FIG. 13 is a graph illustrating the amount of carbohydrates in the gut of the patient of FIG. 11 as a function of time. It should be noted that insulin and meal events are recorded in the figures, but it is also evident, based on the measured data, that the recordation of the insulin and meal events is not complete. This is indicative of one problem with the patient recorded data, that is, the data may be incomplete or inaccurate. As is described herein, various embodiments of the present invention take such incompleteness or inaccurate recording of data into account when generating a patient's predicted future glucose level.

The usefulness of such an analysis can be seen by referring to FIG. 12, for example. In FIG. 12, the graph shows the insulin pharmacokinetics and pharmacodynamics for a patient. Analyzing the difference between these lines may reveal a patient-specific abnormality that would suggest a particular treatment. Accordingly, the parameters of the model, or the assumptions or rule sets included therein, may be adjusted to more accurately predict a patient's glucose level at any future time given the inputs described above.

In another example, for someone not dosing insulin, the model may determine that there maybe residual insulin some time in the future. In such a case, the amount of the residual insulin thus forecast may be used by a clinician to prescribe the use of oral anti-diabetes agents or insulin to control the patient's blood glucose level.

Once the underling physiological state has been identified, the patient's caregiver may access the therapy decisions and either adopt, adapt or modify the patient's care regiment to incorporate these patient specific parameters. For example, referring again to FIG. 11-13, and more specifically to FIG. 12, a physician may determine that a much longer-duration of action of insulin should be programmed into a patient's insulin pump to provide an improved therapeutic regimen. In this example, the amount of the residual insulin forecast by the model incorporating the use of longer acting insulin may be used by the clinician to determine if an anti-diabetes agent or insulin should be prescribed for that patient in addition to the current regime.

Improving Parameter Estimates by Temporal Weighting

Retrospective or real-time treatment calculators that utilize continuous glucose monitoring data as well as other available information, such as information concerning the amount of what a patient had to eat, insulin dosing, and amount of exercise the patient performed, can improve diabetes management. In one embodiment, a retrospective treatment calculator is programmed into the software that operates a processor incorporated in a Continuous Glucose Monitoring device, insulin pump, or it maybe embodied in software installed on a computer that loads appropriate patient data. Such a treatment calculator can be used to aid in the evaluation of a patient's state of diabetes management as well aid in dosing adjustments.

In another embodiment, a real-time treatment calculator may be incorporated into software that controls devices such as a CGM device or a mobile device that has appropriate patient data. Treatment calculators need to estimate parameters associated with the patient model using available data, such as from the CGM device.

In one embodiment, the invention includes a method for the parameter estimation in the presence of signal artifacts that could mislead the estimation process from identifying the proper patient parameters. The term artifact as used herein refers to situation where data from a CGM system contains noise. In some cases, the noise can contain significant characteristics that may prevent the system identification process described above from obtaining a good model fit.

When solving a parameter estimation problem, attempts are made to minimize the residual sum of squares between the model-fit and the experimental measurements. In many cases, it is useful to weight the squared residuals asymmetrically, that is, to set some weights to be greater than one. In theory, one should weight each point by the inverse of the measurement variance. By this it is meant that points are weighted less if they are highly uncertain, that is, they have greater variance.

Using this weighting scheme, however, can be problematic as it may be difficult to estimate the point-to-point measurement variance. As a result, alternative weighting schemes may prove more useful. In one embodiment, as used in accordance with the process of system identification of a dynamic model set forth above, points near events (such as, for example, a meal) which drive the model are weighted more heavily. Possible weighting functions include, but are not limited to: triangular functions, step functions, exponential functions, and Gaussian functions. Such functions are well known by those skilled in the art and will not be described in more detail here. The specific parameterization of each weighting function is open as a tuning parameter which may or may not require fixed input.

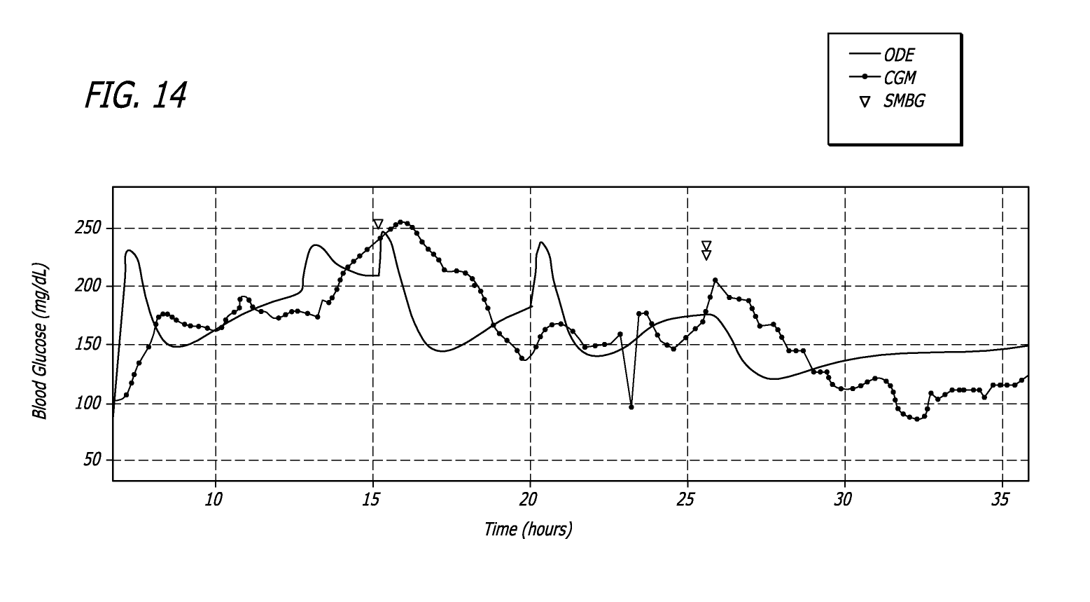

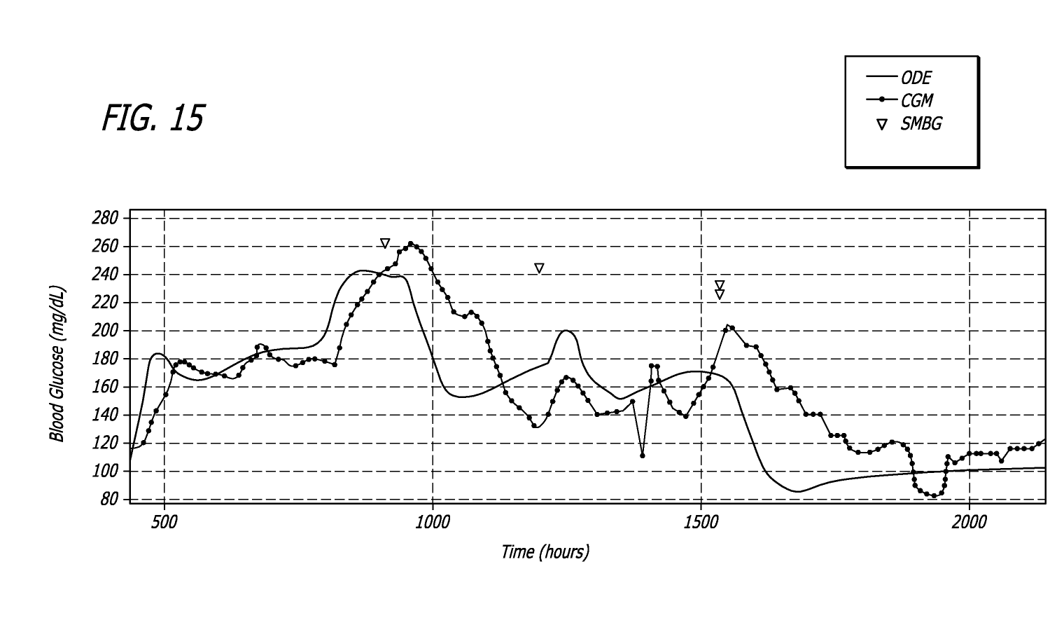

In one embodiment, a model used to describe blood glucose dynamics from CGM data, including data from various meal and insulin events is used to estimate a patient's blood glucose over time. As illustrated in the FIGS. 14-15, dramatic improvement in the qualitative fit of the model estimates compared to data generated using actual CGM data can be obtained by using a triangular weighting function to weight the data representative of each event.

FIG. 14 is a graph showing blood glucose level plotted as a function of time during a representative day for a patient using data taken from a continuous glucose monitoring system along with data predicted using a model using ordinary derivative equations such as set forth above. Also shown are specific glucose levels labeled SMBG taken using a discrete monitoring method, such as a finger stick process. The model fit shows several large transients due to symmetric weight of the fit residuals of the model. For example, the main peaks observed in the CGM data are associated with post-meal peaks, and are not predicted close enough by the fitted model to provide a desired level of the patient's glucose levels.

FIG. 15 is a graph showing the blood glucose data of FIG. 14 plotted against data estimated by the model showing that the model fit is improved by using improved weighting of the model parameters. The model used to generate the ODE line in this graph was adjusted to incorporate a the triangular weighting function. In this model, the triangular weighting function has a value of 10 (ten) at the time of the event which then decays to the nominal weight of 1 (one) after 150 minutes. The relative fit between the CGM data and the glucose levels predicted using the adjusted model is significantly improved.

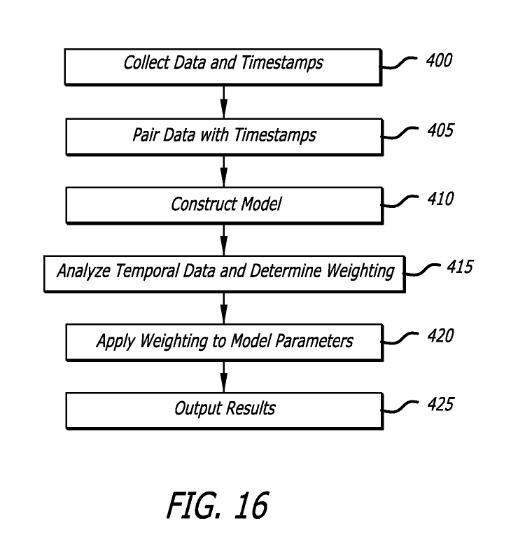

An exemplary process incorporating embodiments of the present invention is illustrated in FIG. 16.

Glucose data and insulin data, such as amount, type, and timestamp of the insulin administration or injection, and meal data, such as amount, timestamp, fat content, etc., and other relevant data are retrieved or accessed by a processor or computer programmed using suitable software commands in box 400.

In box 405, the glucose, insulin, and meal data are paired to the nearest timestamps. For example, if the CGMs are recorded every 10 minutes, and a meal occurs at the 4^{th} minute of the hour, then that meal is paired to the nearest 0^{th} minute of the hour of CGM data. If a meal occurs at the 9^{th} minute of the hour, then that meal is paired to the nearest 10^{th} minute of the hour of CGM data. The insulin data is treated similarly.

Data from each pair is used to construct a row of data for model fitting in box 410. Without loss of generality, suppose, for example, the method of Least-Squares Error regression/fit is used. Then, the regressand and regressors (known to those skilled in the art) can be constructed by placing the proper data from the paired series based on the physiological model used.

As described above, rather than analyzing the data using equal weighting of each regressand-regressor pair, different temporal weighting is applied to each pair. The weighting can be described as follows.

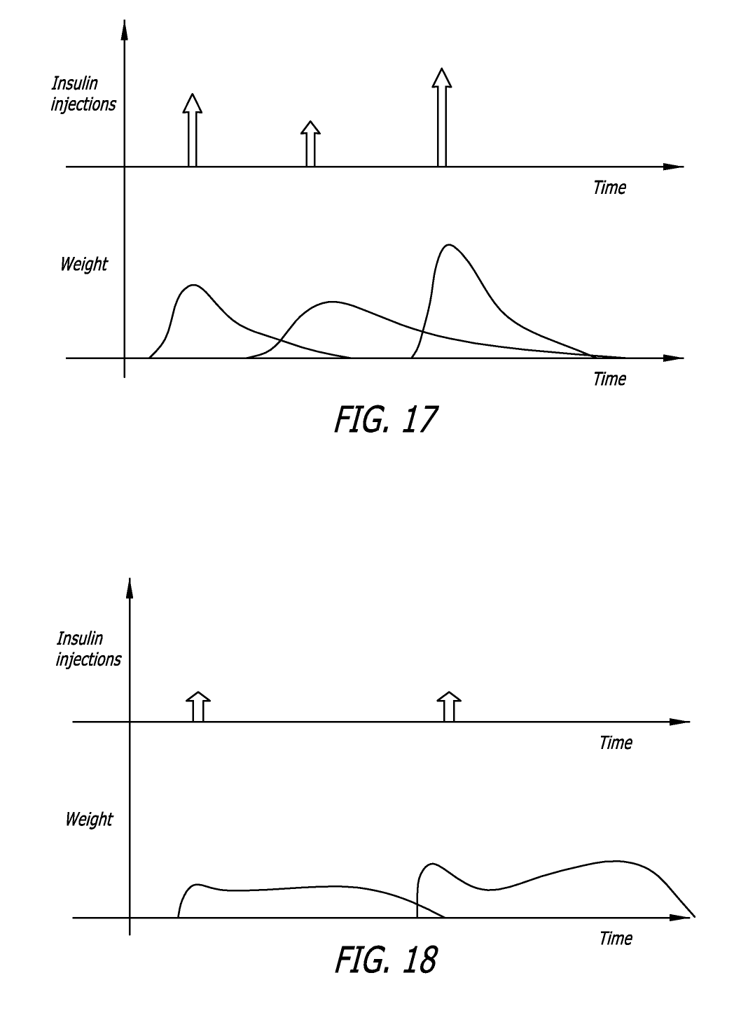

FIG. 17 illustrates how weighting changes the distribution of data associated with an event. In this figure, the relative size of the arrows in the top plot shows the effect on blood glucose caused by insulin injections at different times and in different amounts. Assume for this example that the arrows represent different types of insulin. Then, depending on the type and amount insulin administered, each insulin injection will affect the person's physiology in different ways. A larger dose of insulin would affect more data around the event, while a smaller dose of insulin would affect less data around the event. The bottom plot of FIG. 17 shows the effect of assigning a different weight to each event, where each insulin injection now results in different points in time to become more important in terms of the event's effect on model fitting.

Similar to the insulin injections that act like bolus insulin, long acting insulin that acts like basal insulin can also be used to create a weighting. This is shown in FIG. 18. Once again the top plot shows the injection of the long acting insulin, and the bottom plot shows the effect of the long acting insulin on insulin level when a weighting function has been applied in the model. Application of the weighting functions provides an improved estimation of the actual insulin level in the patient's body.

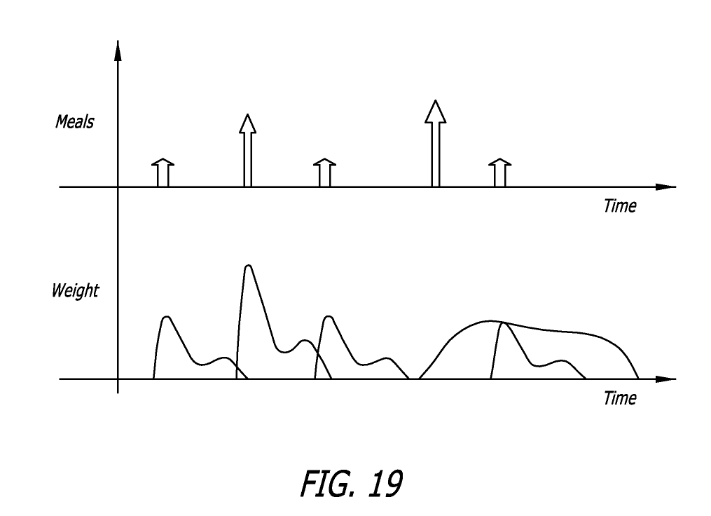

The same process can be used to improve the value of meal information, as shown in FIG. 19. In FIG. 19, each of the meal event data may be assigned a different weight, depending on the amount of food consumed, its composition, and the timing of the meal.

All the weights from the different events, such as insulin and meals, can then be combined in the model. One exemplary method to combine them is to take the direct sum of the weights for every point in time that glucose level data exists. Another method is to take the maximum weight for every point in time that glucose level data exists.

The weights for each point in time are then used to modify the relative importance of one regressor-regressand pair compared to another. One common method to perform this, by way of example, is to use the weights to formulate a weighted least-squares fit.

Referring again to FIG. 16, the temporal data is analyzed and weighting is determined in box 415, and the weighting is applied to the model parameters in box 420. The results of the process are outputted in box 425 for use in making insulin therapy recommendations to the patient. These recommendations may be used to either automatically or manually control an insulin delivery device, or they may cause a display to provide a prompt to a patient to either administer more insulin, or a different type of insulin, or modify an insulin delivery regime, or to consume carbohydrates to prevent hypoglycemia.

Improving Accuracy of Parameter Estimates Using Temporal Event Shifting

In another embodiment, the present invention includes a method for parameter estimation in the presence of practical uncertainties in the accuracy of information, particularly in the accuracy of the time stamp of the information. For example, when a patient enters meal information, or the time of an insulin injection, the patient may enter the time of the event inaccurately.

When solving a parameter estimation problem, a model may be fitted whose qualitative behavior may appear promising though non-ideal. For example, the model may respond to various inputs, increasing or decreasing as expected, but not to the same degree as observed in the actual measurement data that is being fit to the model.

In the case where input events such as insulin dosing, consumption of meals or performance of exercise, are recorded by someone, such as the typical patient, not trained in good laboratory practices (GLP), or by someone who has no strong motivation to accurately time stamp events, an assumption may be made that the event time stamps recorded by the patient are uncertain.

Accurate time stamps on the data are important in fitting the model to the experimental data, as the model is dependant upon the time course of events such as when meals are taken or the amount or the time and duration of exercise. Uncertainty in the time stamps for these events can lead to inaccuracies in the model fit, and thus reduces the accuracy of any predictions made by the mode as to how an event will affect a patient's future glucose level over time.

For example, when trying to fit a model of blood glucose dynamics in a type 1 diabetic to recorded data, it is realistic to assume that, for various reasons, the patient is likely to record a time of an event incorrectly at least occasionally. By taking this occasional inaccuracy in account when estimating model parameters, the model used can produce better parameter estimates.

By allowing the time stamps on events as well as model parameters to be optimized, fit error may be at least partially mitigated, and improved parameter estimates using the models described previously may be generated. However, limits must be selected on how far an event is allowed to “move” from its recorded time function. In addition, a decision must be made whether to perform this optimization jointly with parameter estimation, or after initial parameter estimates have been made. In the latter case, for example, the process may engage in recursive estimation, fitting model parameters, then event times, and repeating until a stopping criterion is satisfied. Moreover, the time stamp may be allowed to vary continuously, or in discrete steps.

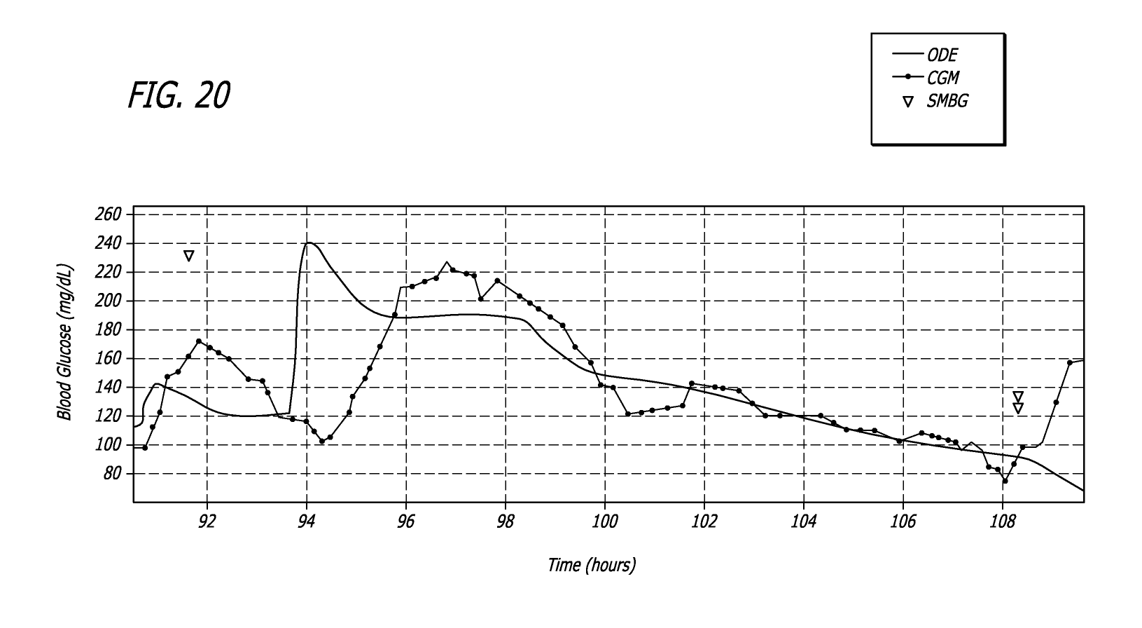

FIG. 20 is a graph showing blood glucose level as a function of time during a representative day for a patient. This graph shows data taken using a continuous glucose monitoring system (CGM) along with data predicted using a model using ordinary derivative equations (ODE) such as set forth above. Additional, specific values labeled SMBG represent data gathered using a discrete glucose measuring method, such a finger stick method. The graph of FIG. 20 shows that the fit of the ODE model data near hour ninety-four time point is significantly different than the actual glucose level as measured by the CGM device.

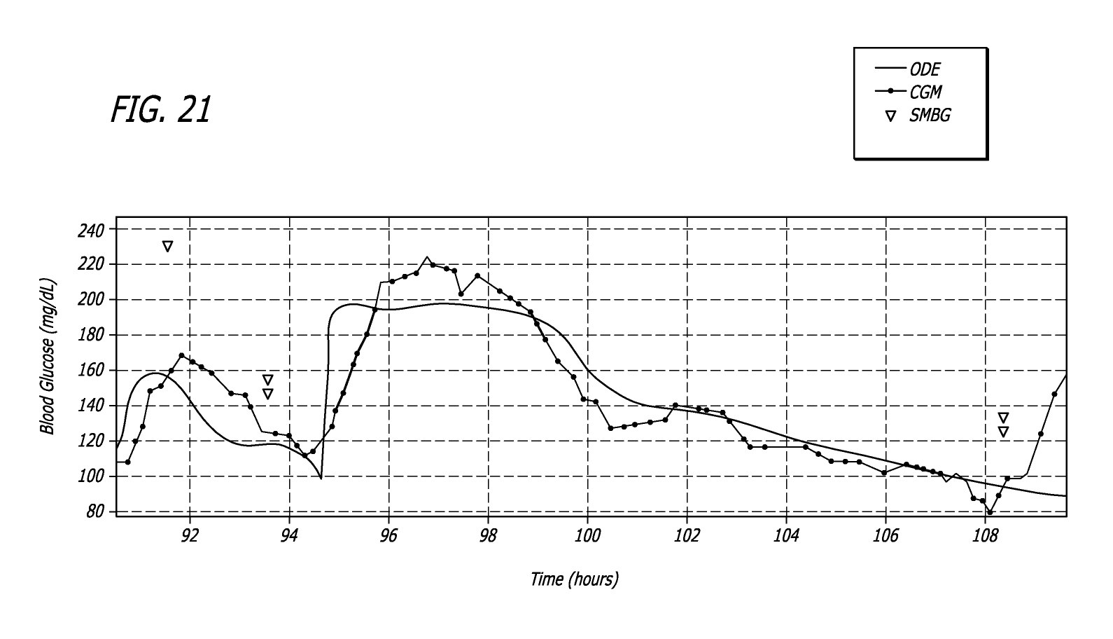

FIG. 21 is a graph similar to that of FIG. 20, except that the ODE model has now incorporated temporal shifting of the meal and insulin events to improve the fit of the model to the actual CGM data. In generating the ODE line in this graph, the model was adjusted to allow meal and insulin events to move to within +/−30 minutes of the recorded times in discrete steps of 10 minutes. In this example, the temporal shifting was performed after the initial parameter events were generated. Comparing the graph of FIG. 21 to FIG. 20 at the 94 hour point shows how use of temporal shifting in the modeling process has significantly improved the fit of the model generated data to the actual CGM data.

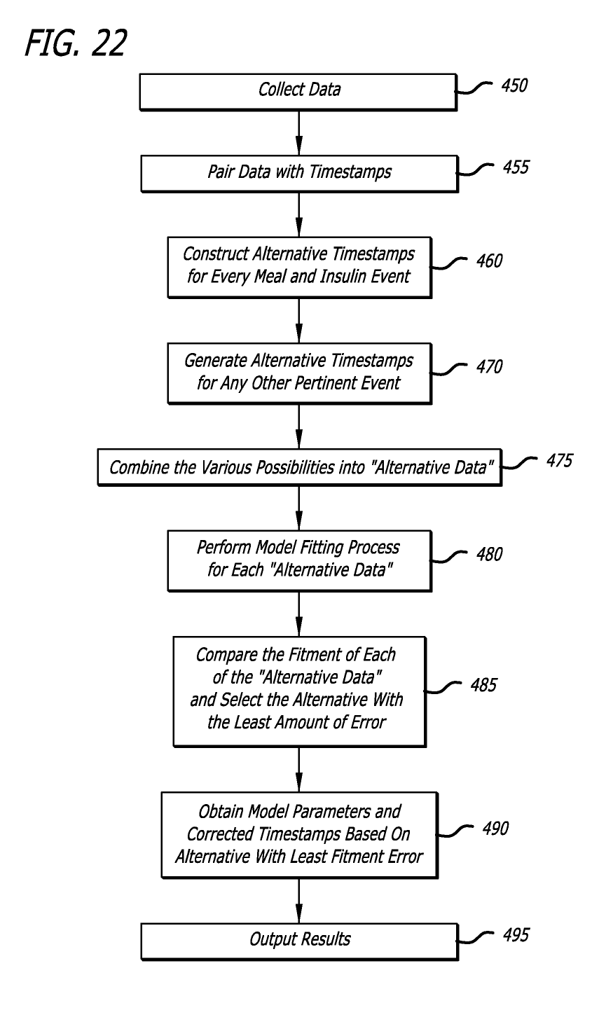

An exemplary process using temporal event shifting to improve model fit is illustrated in the flow chart of FIG. 22. The processes illustrated by this flow chart may be embodied in suitable computer program commands that are used to control a processor to carry out the functions and processes indicated.

Glucose data and insulin data, such as amount, type, and timestamp of the insulin administration or injection, and meal data, such as amount, timestamp, fat content, etc., and other relevant data are retrieved or accessed by a processor or computer programmed using suitable software commands in box 450.

In box 455, the glucose, insulin, and meal data are paired to the nearest timestamps. For example, if the CGMs are recorded every 10 minutes, and a meal occurs at the 4^{th} minute of the hour, then that meal is paired to the nearest 0^{th} minute of the hour of CGM data. If a meal occurs at the 9^{th} minute of the hour, then that meal is paired to the nearest 10^{th} minute of the hour of CGM data. The insulin data is treated similarly.

For every user entered meal and insulin information, it is possible that the timestamp may correspond to the actual start of the event, anticipated start of the event, actual end of the event, recalled start of the event, or other variations. The reason for this is that the timestamp is dependent on the patient's ability to record the events in a consistent manner given their individual circumstances at the time of the event.

Rather than performing the steps set forth in boxes 400-410 of FIG. 16, the timestamps of the insulin and meal events may be varied within a finite window of time. Thus, the process, in box 460, constructs several alternate timestamps for every meal and insulin event. For example, if a meal is recorded to have taken place at 1500 hours, one may consider, for example, seven possibilities: the meal actually started at 1430 hours, or 1440, or 1450, or 1500 (as recorded), or 1510, or 1520, or 1530.

In box 470, the processor generates a similar range of possible timestamps for other events.

The various possibilities are combined into many “alternate data” in box 475. Model parameter fitting is carried out in box 480 for each alternative data. In one embodiment, for example, the steps described with reference to boxes 410-425 of FIG. 16 may be used.

The fitment of each of the alternative data computed in box 475 are compared, and alternative with the least amount of fitment error is selected in box 485. The error metric used in this comparison may be based on whatever parameter fitting method is used. For example, where least-squares error fitting is used, an alternative with the lowest sum of the square of the error may be deemed most suitable.

In box 490, the process obtains the model parameters and corrected timestamps of the meal and insulin events based on the possibility with the least fitment error. The results of the process are output in box 495 for use in providing insulin therapy recommendations to the patient.

Detecting Gastroparesis

As stated previously, the time for emptying the stomach is important for determining the parameters to be used in formulating an accurate model of the insulin dynamics of a specific patient. Gastroparesis is often associated with diabetic neuropathy wherein the emptying of the stomach contents to the small intestines is significantly delayed. Obviously, a delay in emptying of the stomach can seriously effect the estimation of the stomach emptying parameters used in the fitting the model to the field generated data.

Gastroparesis often goes undiagnosed for some time due to a typically mild initial presentation. Complications from gastroparesis include heart burn, nausea, vomiting of undigested food, an early feeling of fullness when eating, weight loss, abdominal bloating, lack of appetite, gastroesophageal reflux, and spasms of the stomach wall. In addition, because of the delay in gastric emptying, prandial insulin action no longer fully coincides with glucose input from the gut, making gut glucose control more difficult. Using continuous glucose monitoring data from a continuous glucose monitoring system, such as the FreeStyle Navigator® Continuous Glucose Monitoring System that is distributed by Abbott Diabetes Care, or a similar device, the rate of gastric emptying among other physiological parameters can be estimated using variations of the above-identified mathematical model and various parameter estimation techniques, including but not limited to: expectation maximization, maximum likelihood estimation, extended Kalman filtering, extended Kalman smoothing, unscented Kalman filtering, unscented Kalman smoothing and unscented Rauch-Tung, Striebel smoothing.

Once the rate of gastric emptying has been calculated, the data may be stored and accessed by the physician to ascertain whether a patient has developed gastroparesis or whether the patient is likely to develop gastroparesis in the near future. Since the gastric entering rate may be continually estimated from continuous glucose data, by examining trends in this estimator, or by setting threshold values, a physician may quantify the progression of the pathology and provide additional support for inconclusive test results. Thus, the embodiment of the method described above provides a quantative indicator that a patient may be developing or may already have developed gastroparesis. Use of this embodiment of the present invention may result in earlier diagnosis of gastroparesis at essentially no additional cost to the patient in time or money for patients using continuous glucose monitors.

To produce a quantitative estimate of gastric health, a patient specific model of an individual patient which accounts for the action of insulin and meals on blood glucose is developed as described above. Using CGM data as well as logs of insulin and meal events, the parameters of the model are identified by determining the parameter or parameters corresponding to the rate of gastric emptying, and the likelihood of gastroparesis is estimated. Furthermore, by tracking the history of the relevant parameters over many measurements and/or physician visits, the development of gastroparesis can be anticipated and the efficacy of its treatment maybe quantitatively assessed.

In one embodiment, for example, a model describing diabetic blood glucose dynamics may use an extension to the Bergman minimal model as described above. For example, the parameter kₑₘₚ in the equation 4 (described above) corresponds to the rate of gastric emptying.

Data generated from a separate offline study may be performed, or results from existing studies may be investigated in order to obtain a distribution of normal versus abnormal gastric emptying rate parameters for the patient. This distribution is then used to diagnose when a person's gastric emptying rate parameter signals gastroparesis. For example, a connection between a known diagnosis of gastroparesis and gastric emptying rate can be made such that a single threshold may be employed. Another example is to evaluate other measurable factors, such as number of years since diagnosed with diabetes, person's age, person's body mass index (BMI), gender, primary diet composition, and the like, such that different gastric emptying rate thresholds may be employed for persons with different ranges of measurable factors.

The various embodiments of the present invention discussed above are advantageous over prior methods of forecasting a patient's future glucose levels. The various embodiments are useful in improving prediction of a patients glucose levels so that the data generated by the models of the various embodiments can be used to provide accurate control of an automatic, closed loop insulin delivery system. Moreover, the various embodiment proved improved methods incorporating adjustments to improve calculation time and to account for inconsistencies in data, such as meal and time of event data, provided by a patient.

While several specific embodiments of the invention have been illustrated an described, it will be apparent that various modifications can be made without departing from the spirit and scope of the invention. Accordingly, it is not intended that the invention be limited, except as by the appended claims.

We claim:

1. A method for predicting future blood glucose values from blood glucose data collected over time for a patient, comprising:
measuring blood glucose data at selected times over a selected sampling period;
collecting data related to insulin delivery, carbohydrate intake and exercise over the selected sampling period;
determining values for selected patient specific parameters from the blood glucose data, insulin delivery and meal data;
providing the determined values to a model to determine a patient's reaction to insulin therapy, carbohydrate intake and exercise;
processing the model to provide a model output;
predicting the patient's future blood glucose values from the model output.

2. The method of claim 1, wherein determining values for selected patient specific parameters and processing the model is carried out by a processor under control of suitable software programming commands.

3. The method of claim 1, wherein the model used is an extended version of the Bergman Minimal Model.

4. The method of claim 1, wherein the model is set up using a pseudo-steady state assumption to simply the calculation requirements of the model.

5. The method of claim 1, wherein the model includes determining an insulin effectiveness as a function of insulin sensitivity and dosage size.

6. The method of claim 1, further comprising
transforming the model output into physiologically meaningful parameters including at least one parameter selected from the group of parameters consisting of insulin pharmacokinetics, insulin pharmacodynamics, residual beta cell function, liver function, gastric function, and counter-regulatory response to low blood and exercise-induced glucagon secretion.

7. The method of claim 6, further comprising:
determining the patient's disease state using the physiologically meaningful parameters.

8. The method of claim 1, further comprising:
providing data related to events such as carbohydrate intake, insulin dosage and duration and intensity of exercise;
temporally weighting such data;
providing the temporally weighted data to the model to improve the correspondence of predicted future glucose values to measured blood glucose data.

9. The method of claim 1, further comprising:
providing data related to events such as carbohydrate intake, insulin dosage and duration and intensity of exercise;
temporally shifting such data;
providing the temporally shifted data to the model to improve the correspondence of the predicted future glucose values to measured blood glucose data.

10. The method of claim 1, wherein the model is simplified using at least one assumption regarding selected data to reduce the time needed to determine the selected parameters.

11. The method of claim 1, further comprising:
determining an insulin sensitivity factor from the model output.

12. The method of claim 1, further comprising:
determining an insulin to carbohydrate ratio from the model output.

13. The method of claim 1, further comprising:
determining a total daily dosage of insulin to cover a patient's basal insulin needs from the model output.

14. The method of claim 1, further comprising:
determining an indicator of gastric emptying from the model output.

15. The method of claim 14, wherein determining an indicator of gastric emptying includes using various parameter estimation techniques.

16. The method of claim 15, wherein at least one of the various parameter estimation technique is a technique selected from the group consisting of expectation maximization, maximum likelihood estimation, extended Kalman Filtering, extended Kalman smoothing, unscented Kalman filtering, unscented Kalman smoothing, and unscented Rauch-Tung-Striebel smoothing.

17. A system for controlling insulin delivery to a patient, comprising:
a glucose monitor for providing glucose level data representative of an amount of glucose in a patient's blood stream;
an input device for inputting carbohydrate intake data;
a processor configured to receive the glucose level data and carbohydrate intake data, the processor programmed to analyze the received glucose level and carbohydrate intake data using a model to predict a future glucose level of the patient, and to provide insulin and carbohydrate intake recommendations based on the predicted future glucose level.

18. The system of claim 17, further comprising an insulin pump in operable communication with the processor, and wherein the insulin recommendations are commands transmitted by the processor to the insulin pump to control the pump to deliver insulin to the patient in accordance with the insulin recommendations.

19. The system of claim 18, wherein the model is an extended Bergman Minimal Model.

20. The system of claim 17, further comprising a memory in operable communication the processor in which glucose level, carbohydrate intake data, predicted glucose level data and recommendations are stored.

21. A system for predicting the future glucose level of a patient based upon patient specific parameters, such as glucose level history, insulin delivery history, carbohydrate intake and exercise history, comprising:
an input device for inputting values of at least one parameter selected from the group consisting of glucose level, carbohydrate intake, insulin type, insulin delivery amount, and exercise intensity and duration;
a memory for storing values related to glucose level history, insulin delivery history, carbohydrate intake and exercise, including inputted values for the at least one parameter selected from the group consisting of glucose level, carbohydrate intake, insulin type, insulin delivery amount, and exercise intensity and duration;
a processor in operable communication with the input device and the memory, the processor programmed retrieve data from the memory to calculate patient specific parameters related to the prediction of a future glucose level of the patent, the processor also programmed to use the calculated patient specific parameters as inputs to a model employing algorithms to produce an output related to a future glucose level of the patent, the processor also programmed to uses rule sets and assumptions to simplify production of the output, and wherein the processor is programmed to transform the retrieved data by weighting the data to improve a quality of the output of the model.
